(19) United States
(12) Patent Application Publication
(10) Pub. No.: US 2023/0174939 A1
(43) Pub. Date: Jun. 8, 2023

(54) NOVEL ANUCLEATED CELLS AS A SOURCE FOR TREATMENT OF PLATELET RICH PLASMA DEPENDENT DISORDERS

(71) Applicant: Stellular Bio, Inc., Watertown, MA (US)

(72) Inventor: Dean Falb, Watertown, MA (US)

(73) Assignee: Stellular Bio, Inc., Watertown, MA (US)

(21) Appl. No.: 17/985,365

(22) Filed: Nov. 11, 2022

Related U.S. Application Data
(63) Continuation of application No. PCT/US21/32353, filed on May 13, 2021.
(60) Provisional application No. 63/024,587, filed on May 14, 2020.
(60) Provisional application No. 63/106,009, filed on Oct. 27, 2020.
(60) Provisional application No. 63/144,033, filed on Feb. 1, 2021.

(51) Int. Cl. C12N 5/078; A61P 17/02; A61P 19/02; A61P 17/14; C07K 14/475

(52) U.S. Cl. CPC C12N 5/0644; A61P 17/02; A61P 19/02; A61P 17/14; C07K 14/475; C12N 2506/03

(57) ABSTRACT

Described herein are methods for treating, repairing or ameliorating diseases, disorders, or injuries related to dry eye, osteoarthritis, tendon, ligament, bone repair, wound healing or wound-healing related disorders, alopecia or in skin rejuvenation or regeneration with platelet-like-cells or variants thereof (PLCs) or derivatives thereof or lysates thereof or the platelet rich plasma (PRP) derived therefrom. Also, described herein are methods for generating platelet rich plasma (PRP) from the PLCs or derivatives thereof or lysates thereof.

### RELATED APPLICATIONS

This application is a continuation of PCT/US21/32353, filed on May 3, 2021, which claims the benefit of and priority to U.S. Provisional Patent Application Ser. No. 63/024,587, filed May 14, 2020; U.S. Provisional Patent Application Ser. No. 63/106,009, filed Oct. 27, 2020, and U.S. Provisional Patent Application Ser. No. 63/144,033, filed Feb. 1, 2021, the entire contents of each of which are herein incorporated by reference in their entireties.

### BACKGROUND

Platelet rich plasma (PRP) has been implicated to play a role in medical treatment of wound healing or speeding repairs of damaged tissues such as cartilage, tendons, ligaments and bone, or in treating dry eye and other diseases. However, there is a paucity of critical data regarding the beneficial effects of PRP in clinical procedures because of limited sample size of reported data from platelet donors. Moreover, every platelet donor is different, therefore, there is no consistency in the quality of PRP produced for medical procedures. PRP, currently in use, is plagued with several other disadvantages, such as batch to batch variability because every patient provides his or her own platelets as a source for his/her PRP. Other disadvantages include the short life-span of donor platelets, impurities or contaminations, or lack of availability or shortages in supply. As a result of a lack of standardization of PRP products, clinical effectiveness of PRP is still under debate. Hence, the field is in dire need of a safe, standardized, defined, off-the-shelf product that could be developed and approved for use in humans by regulatory bodies, instead of having to make separate, unreliable PRP preparations for each patient, which has raised skepticism regarding its potential efficacy and use.

Osteoarthritis (OA) is an example of a disease for which there is a significant unmet need, and a treatment is urgently desired. In the current OA treatment, patients are given NSAID and Duloxetine. If symptoms do not improve, oral duloxetine is started. If after three months, symptoms improve, still nonpharmacological management and oral duloxetine treatment are continued and symptom progression is monitored, which could be a prolonged treatment process. If symptoms persist or progress after three months, intraarticular glucocorticoid has to be injected into patients who require short-term pain relief. If symptoms continue to progress, patients are deemed to require a surgery and are referred to an orthopedic surgeon. Hence, novel, less aggressive and better therapeutic options for the treatment of OA and other diseases with unmet medical needs (e.g., wound healing, dry eye disease, alopecia or damaged or aging skin etc.) are needed and are described in this application.

### SUMMARY

The methods and compositions of the present disclosure advantageously utilize the novel anucleated platelets or platelet-like cells or platelet variants (collectively referred to as “PLCs” (or in its singular form: “PLC”)) or derivatives thereof or lysates thereof or platelet rich plasma derived therefrom to meet unmet needs of treating, repairing or ameliorating diseases, disorders, or injuries related to osteoarthritis, tendon, ligament, bone repair, wound healing or wound-healing related disorders, dry eye, alopecia or skin rejuvenation or regeneration for which no adequate or consistent treatments are available by conventional means, for example by the use of donor platelets.

Advantageously, the PLCs or derivatives thereof or PRP derived therefrom are, consistent in quality and composition and can be made at a relatively lower cost, as compared to that obtained from platelets from a human donor. More importantly, it eliminates the need to extract PRP from a donor's blood, thereby eliminating the need of puncturing a patient to withdraw blood. Moreover, the PLCs are easy to prepare as they come out of a bioreactor or a fluidic device and can be administered in a less aggressive manner than other therapeutic options (e.g., no surgeries or complex medical procedures are involved). The PLCs or derivatives thereof or lysates thereof, produced by the methods of the present disclosure, can be easily scaled up, can be continuous in supply (unhindered by batch to batch variability), are relatively free of contaminants, can be used as supplement a donor derived PRP, can be locally administered at an injury site or in the vicinity thereof, to treat, repair or alleviate tissue injury or tissue injury related diseases, such as but not limited, to bone-related disorders (e.g., osteoarthritis) or dry eye disease, or treat or mitigate a regenerative disease or disorder related to it or as a cosmetic to treat or mitigate aging of skin or loss of hair.

Thus, in some embodiments there are provided compositions comprising the PLCs or derivatives thereof or lysates thereof or PRP derived therefrom or precursor cells making the PLCs or derivatives thereof or PRP derived therefrom that can be administered directly or near the site of tissue damage to accelerate the healing of injured tissues, such as but not limited to, tendons, ligaments, muscles, joints, or other musculoskeletal injuries. In some embodiments, the composition comprising the PLCs or derivatives thereof or PRP derived therefrom or precursor cells making the PLCs or derivatives thereof or PRP derived therefrom are administered locally or topically in and around the vicinity of an injury site (e.g., in the knee for the osteoarthritis treatment) or under an aging skin or in its vicinity for skin or hair regeneration or rejuvenation. In some embodiments, the PLCs or derivatives thereof or precursor cells making the PLCs or derivatives thereof or PRP derived therefrom are in a lysate form i.e., the PLCs or derivatives thereof or precursor cells making the PLCs or derivatives thereof or PRP derived therefrom are concentrated, and lysates are prepared from the concentrates. In some embodiments, the PLCs or the lysates are concentrated and are diluted prior to use in a carrier, a diluent, or a buffer, or a donor-derived PRP as disclosed herein. In some embodiments, patients (i.e., subjects) subjected to such treatment are cancer patients that were subjected to chemotherapy or other cancer-based treatments which leads to tissue injury or damage or hair loss but the treatment by the present disclosure is not for cancer per se. In some embodiments, the treatment is in cancer free patients.

In some embodiments, PLCs or derivatives thereof or lysates thereof are enriched with growth factors or with agents that stimulate the release of growth factors from the PLCs or derivatives thereof or are mixed with the platelet-rich plasma from a subject in a combination that best suits the subject.

The PLCs or derivatives thereof are generated by one or more tools and technologies, such as, bioreactors or fluidic devices. Bioreactors or fluidic devices could include, but is not limited to, shear stress, mechanical strain and pulsed electromagnetic field bioreactors, large-scale stirred tank bioreactors, automated bioreactors, rotating wall bioreactors (RWBs), and rocking motions as seen with wave bioreactors, organ-on-chip bioreactors. Other bioreactor configurations that enable continuous, perfusion operation such as packed bed bioreactors (PBBs), fluidized bed bioreactors (FBBs), or PBBs or FBBs including the use of microcarriers, CultiBag bioreactors, and membrane bioreactors such as hollow fiber bioreactors (HFBs) are also contemplated for generating the PLCs/EVs or derivatives thereof of the present disclosure. Operation of the bioreactors may require coupling with an internal or external cell retention device on a recycle line, by centrifugation, sedimentation, ultrasonic separation or microfiltration with spin-filters, alternating tangential flow (ATF) filtration or tangential flow filtration (TFF) or in vivo bioreactors, which are a pocket within the body into which biomaterials (e.g., PLCs or their derivatives or the progenitor cells form which they are derived from) are implanted at a site in need thereof and incubated for an extended period of time. Within these pockets (for example, bone tissue or muscle flap etc.), the grafts harness the regenerative capacity of the body to recover from a disease or an injury. Non-limiting examples of bioreactors are described, for example, in the co-filed application titled: Simultaneous Welding of Three Components To Form a Bioreactor or Filter Structure (U.S. Application No. 62/981,373) or elsewhere, for example tools and technologies (e.g., bioreactors or fluidic devices) disclosed in U.S. Pat. Nos. 9,795,965; 10,343,163; 9,763,984; 9,993,503; and 10,426,799; US Publication No. 20180334652; PCT Applications PCT/US2018/021354; PCT/US2019/012437, PCT/US19/040021 and U.S. application Ser. No. 16/730,603, each of which is incorporated herein in their entirety by reference. Bioreactors or microfluidic devices known or unknown that can routinely generate the PLCs or derivatives are also contemplated for use in the present disclosure.

In some embodiments, the source of the PLCs or derivatives thereof or lysates derived thereof or PRP derived therefrom or precursor cells making the PLCs or derivatives thereof or PRP derived therefrom are autologous, i.e., that they are produced utilizing, for example, the CD34+ progenitor cells of an individual in need of the PLC-based treatment, wherein the progenitor cells are cultured through a bioreactor to produce PLCs or derivatives thereof, which can be used as such, i.e., as PLCs or derivatives thereof or lysates thereof or PRP derived therefrom (i.e., PRP enriched with the PLCs). In some embodiments, the source of the PLCs or derivatives thereof or lysates thereof or PRP derived therefrom are iPSCs or the megakaryocytes derived from the iPSCs.

Several other advantages provided by the methods and compositions comprising the PLCs or derivatives thereof or lysates thereof or PRP derived therefrom are that they are essentially allogeneic, which minimizes the risk of immune responses, are not cancerous, i.e., do not exhibit uncontrolled growth or tumor formation in vivo, and are enriched in growth factors, such as but not limited to, fibronectin, vitronectin, sphingosine 1-phosphate, which facilitate the wound healing process. PLCs or derivatives thereof or lysates thereof or PRP derived therefrom or precursor cells making the PLCs or derivatives thereof or lysates thereof or PRP derived therefrom may also reduce or eliminate detrimental side-effects elicited by anti-inflammatory agents, opioids, or other drugs, for example.

In some embodiments, the present disclosure provides a method of treating a subject suffering from an injury, the method comprising administering to the subject in therapeutic amounts a composition comprising PLCs or derivatives thereof or lysates thereof or the PRP derived therefrom of the present disclosure thereby causing amelioration of or treatment or repair of the injuries (e.g., osteoarthritis, tendon, ligament, bone repair, wound healing or wound-healing related disorders) or assist in regeneration/rejuvenation of skin or hair (alopecia). In some embodiments, the method comprises administering a second or a third therapeutic agent.

In some embodiments, the present disclosure provides a pharmaceutical composition comprising the PLCs or derivatives thereof or lysates thereof or PRP derived therefrom or the PLCs or derivatives of the PLCs or precursor cells making the PLCs or derivatives thereof or lysates thereof or PRP derived therefrom and one or more pharmaceutically acceptable bulking agent, a carrier or excipient. In some embodiments, the pharmaceutical composition further comprises a second or a third therapeutic agent.

In some embodiments, the present disclosure provides non-natural extracellular vesicles (EVs) that are made in vitro as admixtures with the PLCs. EVs comprise microvesicles (MV) and exosomes, 200-1000 nm in diameter, carrying biologically active multifarious molecules such as proteins, lipids, and RNAs either on their surface or within their lumen. Each component in the admixture, i.e., PLCs, microvesicles and exosomes can substantially be isolated into individual components from the admixture, for example based on their size. The extracellular vesicles (EVs) are implicated to play a role in stimulating wound healings, regenerating bone tissue to influence the process of bone regeneration and repair or in skin or hair regeneration, indicating that they can confer, for example, wound healing, tissue regeneration, antiapoptotic or anti-inflammatory actions through transporting RNA and protein cargos. They also function as a transport and delivery system for bioactive molecules, play a role in hemostasis and thrombosis, inflammation, malignancy infection transfer, angiogenesis, and immunity. Thus, in some embodiments, EVs may complement PLCs or their derivatives and their combinational use is an even richer resource for PLC-based therapeutic applications.

In some embodiments, the EVs of the present disclosure comprise exosomes, approximately ranging between 65 nm to about 10 μm in diameter carrying multifarious molecules such as proteins, lipids, and RNAs either on their surface or within their lumen. Exosomes play a role in stimulating tissue regeneration, in many in vitro and in vivo models, demonstrating that they can confer proangiogenic, proliferative, antiapoptotic and anti-inflammatory actions through transporting RNA and protein cargos. Thus, in some embodiments, exosomes make it even a richer resource for PLC-based therapeutic applications. In some embodiments, PLCs or derivatives thereof or lysates thereof or PRP derived therefrom or the PLCs or derivatives of the PLCs or precursor cells making the PLCs or derivatives thereof or PRP derived therefrom are administered in combination with the extracellular vesicles (EVs) that are produced as admixtures with the PLCs but can substantially be isolated from the PLCs based on their smaller size. Thus, in an aspect, the microvesicles or the exosomes, alone or in combination with the PLCs or derivatives thereof, can be used for stimulating wound healings, regenerating bone tissue to influence the process of bone regeneration and repair or in skin or hair regeneration, for example, through actions such as transporting RNA or transporting agents (e.g., wound healing agents, tissue regeneration agents, antiapoptotic agents, anti-inflammatory agents or the like) or protein cargos, thereby making them even a richer resource for PLC-based therapeutic applications to confer proangiogenic, proliferative, antiapoptotic or anti-inflammatory effects that may be desired in a patient undergoing PLC-based treatment as disclosed herein.

In some embodiments, the megakaryocytic progenitors, megakaryocytes, proplatelets, preplatelets derived from induced pluripotent stem cells (iPSCs), which produce the platelet like cells (PLCs) and EVs (i.e., microvesicles or exosomes or a combination thereof), prior to passaging through a bioreactor or a fluidic device, can be genetically engineered to express a nucleic acid encoding a protein of interest (for example, wound healing agents, tissue regeneration agents, antiapoptotic agents, anti-inflammatory agents or the like). In some embodiments, PLC and/or EVs can be genetically engineered once such cells were subjected to a passage through the bioreactor or a fluidic device. Thus, in some embodiments, genetic modifications can take place at the stem cell level, in megakaryocytes or in some embodiments in the PLCs and/or the EVs or at any other level during the generation of PLCs and/or the EVs that accompany the PLC and/or EV production. Genetic engineering of megakaryocytes or megakaryocytic progenitors differentiated from a genetically engineered human pluripotent stem cells (hPSCs) cell or cell lines, where the genetic manipulation leads to megakaryocytes or megakaryocytic progenitor cells to express a protein or a polypeptide of interest are also contemplated by the present disclosure. In some embodiments, the PLCs and/or EVs or derivatives thereof, differentiated from the genetically engineered progenitor cells (e.g., megakaryocytes or megakaryocytic progenitor cells), deliver a protein of interest (e.g., wound healing agents, tissue regeneration agents, antiapoptotic agents, anti-inflammatory agents or the like) systemically or at first diseased location, generally the site of a disease where the PLCs and/or the EVs (or genetically engineered versions thereof) are administered, or to a second diseased location, different from the site where the PLCs and/or EVs or derivatives thereof are administered. Examples of such genetically engineered induced pluripotent stem cells or PSC-derived megakaryocytes that produce the PLCs and/or EVs (i.e., genetically engineered PLCs/EVs or derivatives thereof) are disclosed in co-pending U.S. patent application Ser. Nos. 17/213,552 and 17/213,796, respectively, incorporated herein in their entireties by reference. Thus, in some embodiments, genetically engineered PLCs (ePLCs) may be produced by genetically engineered PLC-producing progenitor cells such that ePLCs express an exogenous gene of interest for example for wound healing agents, tissue regeneration agents, antiapoptotic agents, anti-inflammatory agents, anti-hormonal agents or immunomodulatory agents or the like, enrichment of which will broadly complement the PLC-based treatments.

It may be desirable to administer to the patient other compounds, such as but not limited to a corticosteroid, tetra-substituted pyrimidopyrimidine, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the present disclosure are especially useful for the treatment of immunoinflammatory disorders in combination with other agents—either biologics or small molecules—that modulate the immune response to positively affect disease treatments. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNF alpha. Agents that inhibit TNF alpha include etanercept, adelimumab, infliximab, and CDP-870. In this example (that of agents blocking the effect of TNF alpha), the combination therapy reduces the production of cytokines, etanercept or infliximab act on the remaining fraction of inflammatory cytokines, providing enhanced treatment. Small molecule immunomodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib. Advantageously, when one or more of these compounds are administered with the PLCs or derivatives therefrom, it is expected that the concentration of doses of co-administered compounds, if included in a co-therapy, will be reduced or lowered, thereby reducing any harmful known and unknown side-effect induced by the co-administered compounds if they were administered without the PLCs.

In some embodiments, the present disclosure provides kit comprising the PLCs or derivatives thereof or lysates thereof or PRP derived therefrom or precursor cells making the PLCs or derivatives thereof or PRP derived therefrom or derivatives of the PLCs of the present disclosure.

In some embodiments, the present disclosure provides a composition for treating a condition in a subject, the composition comprising platelet-like cells (PLCs) or derivatives thereof and platelet rich plasma (PRP) derived from the subject. In some embodiments, the composition further comprises a wound healing agent, a tissue regeneration agent, an antiapoptotic agent, an anti-inflammatory agent, anti-hormonal agent or an immunomodulatory agent or a combination thereof. In some embodiments, the composition is diluted to a physiological concentration in a carrier, wherein the carrier comprises a diluent or an excipient. In some embodiments, the carrier is a plasma or a plasma substitute or a plasmalyte. In some embodiments, the PLCs or derivatives thereof are free of red blood cells or hemoglobin content or white blood cells. In some embodiments, the composition further comprises extracellular vesicles (EV). In some embodiments, the composition is formulated for application to a site of injury for therapeutic use. In some embodiments, the composition further comprises another therapeutic agent.

### BRIEF DESCRIPTION OF FIGURES

The present disclosure will be described in the detailed description which follows, in reference to the noted plurality of drawings by way of non-limiting examples of exemplary embodiments, in which like reference numerals represent similar parts throughout the several views of the drawings, and wherein:

FIGS. 1A-1B show growth factor and angiogenesis factor profile in PLCs as compared to donor platelets.

FIG. 2 shows a schematic design of PLC treatment for osteoarthritis in rats.

FIGS. 3A-3F show osteoarthritis-induced rats, when treated with PLCs, make strong recovery from osteoarthritis in about 8 weeks after treatment with PLCs as compared to untreated animals. Rats received a single dose at day 18. FIGS. 3A-3B show PLC treated rats after 2 weeks of treatment. FIGS. 3C-3D shows PLC treated rats after 4 weeks of treatment. FIGS. 3E-3F show PLC treated rats after 8 weeks of treatment.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; plasma or plasma substitutes, dextran and hydroxyethyl starch, perfluorocarbons and stroma-free hemoglobin; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

The term “derivatives”, as used herein, refer to genetically engineered PLCs (ePLCs) or genetically engineered extracellular vesicles (eEVs) or a combination thereof for therapeutic use, inclusive of PLC precursor cells (e.g., pluripotent stem cells genetically engineered in a manner such that that the PLCs or extracellular vesicles produced by these PLC/EV precursor cells produce a molecule of interest in the PLCs or extracellular vesicles or in both, or in any other modification described herein. Derivatives are also inclusive of bioconjugates of PLCs and extracellular vesicles or bioconjugates of genetically engineered PLCs and extracellular vesicles. Derivatives are also inclusive of cargo carrying PLCs and extracellular vesicles or cargo carrying genetically engineered PLCs and extracellular vesicles. Here, for example, the PLCs or extracellular vesicles can be first subjected to genetic engineering, then their cargo carrying capacity is utilized. In other words, the term derivative is inclusive of any modification, genetic, chemical or a combination thereof otherwise of the PLCs, genetically engineered PLC, extracellular vesicles or genetically engineered extracellular vesicles.

As used herein “progenitor cells” refers to IPSC-derived cells, such as preMKs, MKs, proplatelets, preplatelets. It is also inclusive of “pluripotent stem cells”, which includes embryonic stem cells, embryo-derived stem cells, and induced pluripotent stem cells and other stem cells having the capacity to form cells from all three germ layers of the body, regardless of the method by which the pluripotent stem cells are derived. Pluripotent stem cells are defined functionally as stem cells that can have one or more of the following characteristics: (a) be capable of inducing teratomas when transplanted in immunodeficient (SCID) rats; (b) capable of differentiating to cell types of all three germ layers (e.g., can differentiate to ectodermal, mesodermal, and endodermal cell types); or (c) express one or more markers of embryonic stem cells (e.g., express Oct 4, alkaline phosphatase, SSEA-3 surface antigen, SSEA-4 surface antigen, SSEA-5 surface antigen, Nanog, TRA-1-60, TRA-1-81, SOX2, or REX1). Progenitor cells also include “megakaryocytic progenitor” (preMK), which refers to a mononuclear hematopoietic cell that is committed to the megakaryocyte lineage and is a precursor to mature megakaryocytes. Megakaryocytic progenitors are normally found in (but not limited to) bone marrow and other hematopoietic locations, but can also be generated from pluripotent stem cells, such as by further differentiation of hemogenic endothelial cells that were themselves derived from pluripotent stem cells.

The term “megakaryocytic progenitor” (preMK), as used herein, refers to a mononuclear hematopoietic cell that is committed to the megakaryocyte lineage and is a precursor to mature megakaryocytes. Megakaryocytic progenitors are normally found in (but not limited to) bone marrow and other hematopoietic locations, but can also be generated from pluripotent stem cells, such as by further differentiation of hemogenic endothelial cells that were themselves derived from pluripotent stem cells.

The term “induced pluripotent stem cells” (iPS cells or iPSCs) refers to a type of pluripotent stem cell generated by reprogramming a somatic cell by expressing a combination of reprogramming factors. The iPSCs can be generated using fetal, postnatal, newborn, juvenile, or adult somatic cells. Factors that can be used to reprogram somatic cells to pluripotent stem cells include, for example, a combination of Oct 4 (sometimes referred to as Oct 3/4), Sox2, c-Myc, and Klf4. In some embodiments, factors that can be used to reprogram somatic cells to pluripotent stem cells include, for example, a combination of Oct 4, Sox2, Nanog, and Lin28. In some embodiments, at least two, three, or four reprogramming factors are expressed in a somatic cell to reprogram the somatic cell.

“Agonist Activated” cell receptor or ligand activation induced by a receptor specific agonist. Agonists activate cells by binding to their respective receptors or ligands on a cell.

“Variant” as used herein refers to manifesting structural variety, structural deviation, or structural differences. Variants also include PLCs or derivatives thereof or the PLCs or the microsome, exosomes, vesicles or any other product generated by culturing the megakaryocytes in a bioreactor.

“PLC” or “PLCs” or artificial platelets as interchangeably used herein, refer to non-naturally existing, novel, anucleated platelets or platelet-like cells that structurally differ from naturally existing bone marrow derived platelets (i.e., natural counterpart). PLC or PLCs are also inclusive of platelet variants, defined elsewhere.

As used herein, “Variant” or “Variants” as interchangeably used herein refers to manifesting structural variety, structural deviation, or structural differences between PLCs and donor platelets. As non-limited examples, variant comprises greater than an average of 2% CD63 receptors (i.e., CD63^{>average2%}) as compared to the reference resting bone marrow derived platelet cells with less than an average 2% CD63 receptor i.e., (CD63^{<average 2%}). In some embodiments, a variant comprises less than 10% on an average of CD36 receptor (i.e., CD36^{<average80%}) as compared to the reference resting bone marrow derived platelet cells with greater than an average 80% CD36 receptor i.e., (CD36^{>average 80%}); or a variant comprising less than an average of 95% CD42b receptor (i.e., CD42b^{<average95%}) as compared to the reference resting bone marrow derived platelet cells with greater than an average 95% CD42b receptor i.e., (CD42b^{>average 95%}); or a variant comprising less than an average of 90% glycoprotein VI receptor or less i.e., (GPVI^{<average90%}) as compared to the reference resting bone marrow derived platelet cells with greater than an average 90% GPVI receptor i.e., (GPVI^{>average 90%}). The term variant is also inclusive of a structural makeup of the PLCs that is comparable to the structural make-up of naturally existing bone marrow derived platelets, either in resting or in their activated stages. For example, the PLCs and the donor platelets may have m % CD36, or n % CD42a, or o % CD42a-b-d, or p % CD61, or q % CD62p, or x % CD63 receptors, where the m %, n %, o %, p %, q % x % are the same (i.e., have equal values) between the PLCs and the and bone marrow derived platelets. In other words, structurally PLCs may be identical to donor platelets, yet manifest the advantages of the PLC variants disclosed herein in the present disclosure.

By, “comprises,” “comprising,” “containing” and “having” and the like can have the meaning ascribed to them in U.S. patent law and can mean “includes,” “including,” and the like (.g., a composition “comprising” X may consist exclusively of X or may include something additional, e.g., X+Y); “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

“Average” as used herein is a number expressing the central or typical value in a set of data, in particular the mode, median, or (most commonly) the mean, which is calculated by dividing the sum of the values in the set by their number. It also refers to a single value (such as a mean, mode, or median) that summarizes or represents the general significance of a set of unequal values.

“Non-natural” as used herein refers to manufactured, created, or constructed by human beings, artificial, or mimicking something that exists in nature.

Routes of administration for the various embodiments include, but are not limited to, local at a site in need of treatment (e.g., knee in osteoarthritis treatment), topical, transdermal, nasal, and systemic administration (such as, intravenous, intraarterial, intramuscular, subcutaneous, inhalation, rectal, buccal, vaginal, intraperitoneal, intraarticular, ophthalmic, otic, or oral administration). As used herein, “systemic administration” refers to all nondermal routes of administration, and specifically excludes topical and transdermal routes of administration. Additional ways for administering the PLCs or derivatives thereof of the present disclosure include, intrathecal, intracapsular, intraorbital, intracardiac, intradermal, intraperitoneal, transtracheal, subcutaneous, subcuticular, intraarticular, subcapsular, subarachnoid, intraspinal and intersternal injection and infusion.

The term “antagonist” as used herein refers to a substance which initiates a physiological response when combined with a receptor. Agonists activate cells by binding to their respective receptors or ligands on a cell. It can be any agent or entity capable of inhibiting the expression or activity of a protein, polypeptide portion thereof, or polynucleotide. Thus, the antagonist may operate to prevent transcription, translation, post-transcriptional or post-translational processing or otherwise inhibit the activity of the protein, polypeptide, or polynucleotide in any way, via either direct or indirect action. The antagonist may for example be a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide, or polynucleotide itself. Similarly, the antagonist may affect the activity of molecules which are themselves subject to the regulation or modulation by the protein, polypeptide of polynucleotide. The term “Agonist Activated”, as used herein, refers to cell receptor or ligand activation induced by a receptor specific agonist.

“Donor platelets” refer to bone marrow derived platelets physiologically generated in a mammalian (e.g., human) body. Donor PRP refers to PRP prepared from a donor's blood.

The term “subject’ as used herein refers to a mammal, such as but not limited to human, porcine, equine, dogs or cats or any other animal capable of suffering from viral infection. It is inclusive of a healthy population, a population potentially infected with a pathogen or a patient suffering from a viral infection.

The term “drug”, “agent” or “compound” as used herein refers to a biological product or a chemical entity or combination of biological product and chemical entities, administered in a therapeutic amount to a person to treat or prevent or control a disease or condition. The biological product or the chemical entity is inclusive of but not limited to, antibodies or fragments thereof, a low molecular weight compound, but may also be a larger compound, for example, an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof.

The term “agent” “therapeutic composition,” or “therapeutic agent” can be used interchangeably and refers to a therapeutic agent. Agent may be selected from one or more of proteins; peptides; aptamers; antibodies; or fragments thereof; chemicals; small molecules; nucleic acid sequences; nucleic acid analogues. A nucleic acid sequence may be RNA or DNA, and may be single or double stranded, and can be selected from; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro-RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins, and fragments thereof. The agent may be applied to the media, where it contacts the cell and induces its effects. Alternatively, the agent may be intracellular within the cell because of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally occurring non-proteinaceous entities. In some embodiments the agent is a small molecule having a chemical moiety. For example, chemical moieties included unsubstituted or substituted alkyl, aromatic, or heterocyclyl moieties including macrolides, leptomycin and related natural products or analogues thereof. Agents can be known to have a desired activity and/or property or can be selected from a library of diverse compounds.

The term “antibody,” as used herein, refers to an immunoglobulin molecule which specifically binds with an antigen. The term “antibody fragment” refers to a portion of an intact antibody and refers to the antigenic determining variable regions of an intact antibody.

The term “culture conditions” or a “culture medium” or “medium” can be used interchangeably and refers to a medium for culturing cells containing nutrients that maintain cell viability and support cell expansion and maintenance stage or the cell differentiation stage. The cell culture medium, in addition to the embodiments disclosed herein, may contain any of the following in an appropriate combination: salt(s), buffer(s), amino acids, glucose or other sugar(s), antibiotics, serum or serum replacement, and other components such as peptide growth factors, etc. The appropriate cell culture media, for a cell type, is known to those skilled in the art.

As used herein, the terms “treat,” “treating,” “treatment,” and the like refer to reducing or ameliorating a disorder and/or symptom associated therewith. It will be appreciated that, although not precluded, treating a disorder or condition does not require that the disorder, condition, or symptoms associated therewith be eliminated.

The term “PLC-enriched plasma” as used herein refers to the enriched PRP provided by a combination of intact PLCs or derivatives thereof or precursor cells making the PLCs or derivatives, lysates extracted therefrom to enrich other source of plasma sources, such as a donor-derived PRP. Optionally, it could include growth factors, cytokines or other agents from other sources, which complement the therapeutic applications of the PLCs or derivatives thereof.

Specifically, stated, or obvious from context, as used herein, the term “or” is understood to be inclusive. Unless specifically stated or obvious from context, as used herein, the terms “a”, “an”, and “the” are understood to be singular or plural.

The word “substantially” does not exclude “completely” e.g., a composition which is “substantially free” from Y may be completely free from Y. Where necessary, the word “substantially” may be omitted from the definition of the disclosure.

The term “extracellular vesicles (EVs or EV)” as used herein collectively refers to microvesicles and exosomes and generally are very small (generally around 1 micron or less in diameter; microvesicles, generally about 200-1500 nm or less in diameter; exosomes generally about 20-200 nm or less in diameter) phospholipid vesicle shed from a megakaryocyte or other cell. Extracellular vesicles (EVs) may contain or may transport materials such as but not limited to nucleic acids (e.g., siRNA), growth factors, proteins or exogenous genetic materials (e.g., for gene therapy) and express the extracellular markers of their parental cells. Megakaryocyte-derived extracellular vesicles (EVs) may have a role in multiple pathways, including hemostasis and inflammation, and in treating various disorders, such as but not limited to, malignancies (e.g., neoplasia), Alzheimer, and tumor progression and development.

The term “cryopreservation medium” refers to a liquid medium (solution or suspension), capable of preserving structure and metabolism of isolated cells against injury associated with freezing events either within or exterior to the cells and that is safe for infusion or injection into humans. The term further refers to a medium (solution or suspension) containing components, including cryopreservation agents, also determined or known to be safe for human infusion or injection. Preferably, the medium (solution or suspension), and agents, components or elements of the medium are approved by a United States regulatory agency for infusion or injection into humans, e.g., histidine (25-50 mM). “Cryoprotective agents” are agents that are capable of conferring a degree of cryoprotection to cell structure and metabolism upon freezing. Cryoprotective agents within the scope of the present disclosure include arabinogalactan and biological and functional equivalents thereof, glycerol, propylene glycol, and albumin, e.g., human serum albumin, plasma or serum.

The term “therapy” is intended to encompass any form of treatment, prevention, or diagnosis, and includes treatments to both cure and prevent disease. Thus, treatment of a healthy patient is to be considered as therapy. Therapy also covers the alleviation of symptoms, in addition to curative treatments for a disease.

The compositions and methods of the present disclosure take advantage of properties of PLCs or derivatives thereof (including that of engineered PLCs) such as but not limited to providing growth factors, cytokines and other agents to offer a unique opportunity to maximize therapeutic outcomes as well as minimizing side effects in treating, repairing or ameliorating diseases, disorders, or injuries related to osteoarthritis, tendon, ligament, bone repair, wound healing or wound-healing related disorders, dry eye, alopecia or skin rejuvenation or regeneration for which no adequate or consistent treatments are available by convention means.

Prior art protocol for PRP enrichment for its use is essentially performed in multiples steps thereby increasing the risk of contamination and leading to inconsistencies an unreliability of the product. For example, typical preparation off PRP includes multiple steps: (1) a small amount of venous blood (15-50 mL) is drawn from the patient's arm in anticoagulant-containing tubes; (2) the recommended temperature during processing is 21° C.-24° C. to prevent platelet activation during centrifugation of the blood; (3) the blood is centrifuged at 1,200 rpm for 12 minutes; (4) the blood separates into three layers: an upper layer that contains platelets and white blood cells, an intermediate thin layer (the buffy coat) that is rich in white blood cells, and a bottom layer that contains red blood cells; (5) the upper and intermediate buffy layers are transferred to an empty sterile tube. The plasma is centrifuged again at 3,300 rpm for 7 minutes to help with the formation of soft pellets (erythrocytes and platelets) at the bottom of the tube; (6) the upper two-thirds of the plasma is discarded because it is platelet-poor plasma; (7) pellets are homogenized in the lower third (5 mL) of the plasma to create the PRP ready for injection.

The present disclosure eliminates many of the prior art steps involved in PRP preparation thereby minimizing risk of contamination or impurities. Most importantly, it eliminates the need to draw venous blood, often a source of contamination (e.g., viruses, such as HIV) unless analyzed critically. In other words, the present disclosure, at the very least, eliminates the need of drawing blood or the separation of blood into different components before platelets can isolated. This is accomplished by culturing a population of progenitor stem cells until conditions such that they substantially differentiate into matured megakaryocytes. Next, the matured megakaryocytes are cultured in a bioreactor or a fluidic device, wherein a bioreactor gradient in the bioreactor mimics endogenous platelet producing environment to generate PLCs (PLCs) or derivatives thereof. The PLCs or derivatives thereof are then collected from the bioreactor in an amount sufficient to use the PLCs or their derivatives. In some embodiments, the PLCs or their derivatives or lysates thereof can be used on their own or in combination with the EVs. In some embodiments, the PLCs or derivatives thereof or lysates thereof can be used to enrich the donor platelets in a platelet rich plasma (i.e., PLCs or genetically engineered PLCs are mixed with donor-based PRP). In some embodiments, PLCs or derivatives thereof can be used in combination with the EVS or with other drugs disclosed herein.

The PLCs or derivatives thereof or lysates thereof or PRP derived therefrom or precursor cells making the PLCs or derivatives thereof or PRP derived therefrom, which are rich in growth factor and cytokines, can be used in their intact form or in the form of a liquid, a paste, or can be admixed with other agents, such as but not limited to gel, ointment, creams or other emulsifying agents, acceptable diluent, carrier or excipient.

In some embodiments, the present disclosure further provides pharmaceutical composition comprising PLCs or derivatives thereof or lysates thereof or PRP derived therefrom or PLCs or PLC derivatives or precursor cells making the PLCs or derivatives thereof or lysates thereof or PRP derived therefrom of the present disclosure. In some embodiments, a pharmaceutical composition comprises (1) the PLCs or derivatives thereof or lysates thereof or PRP derived therefrom or the PLCs or PLC derivatives or precursor cells making the PLCs or derivatives thereof or PRP derived therefrom, and (2) a pharmaceutically acceptable bulking agent, carrier or excipient. In some embodiments, a pharmaceutical composition comprises (1) the PLCs or derivatives thereof or lysates thereof or PRP derived therefrom or the PLCs or PLC derivatives or precursor cells making the PLCs or derivatives thereof or PRP derived therefrom, (2) a pharmaceutically acceptable bulking agent, carrier or excipient, and optionally, (3) at least one additional therapeutic agent. In some embodiments, a pharmaceutical composition comprises (1) the PLCs or derivatives thereof or lysates thereof or PRP derived therefrom or the PLCs or PLC derivatives or precursor cells making the PLCs or derivatives thereof or PRP derived therefrom, (2) a pharmaceutically acceptable bulking agent, carrier or excipient, and optionally, (3) at least one additional therapeutic agent and an enriching agent.

The composition comprising the PLCs or derivatives thereof or lysates thereof or PRP derived therefrom could advantageously be enriched with or formulated with one or more of a polysaccharide, e.g., a natural polysaccharide (such as hyaluronic acid, hydroxypropylcellulose, karya gum (KG), guar gum (GUG), or gellan gum (GEG)), dermal fillers (such as, Juvéderm®, Juvéderm® Ultra Plus, Perlane®, Belotero®, Restylane®), a semi-synthetic polysaccharide or a synthetic polysaccharide, and a synthetic polymer, (e.g., poly(7-oxanorbornene-2-carboxylate), F127 or poly(lactic-co-glycolic acid) (PLGA)), sodium citrate, calcium chloride, proteoglycans, adenine, guanine, cytosine, thiamine, progenitor stem cells or their derivatives, vitamins, retinols, retinoic acid, retinol palmitate, acetate (e.g.,) tocoferil acetate, a phosphate (e.g., sodium ascorbyl phosphate), D-panthenol, peptides, recombinant growth factors, micronized human-identical hormones, amino acids, phyto-extracts, anti-oxidants, lipoic acid, DMAE, collagen, GAG, trace elements, minerals, proteases, ceramides, polysaccharides, algae, marine extracts, monocytes or a combination thereof.

Additional therapeutic agents include, but are not limited to, those described elsewhere in the present disclosure. The composition comprising the PLCs or derivatives thereof or lysates thereof or PRP derived therefrom or PLCs or PLC derivatives or precursor cells making the PLCs or derivatives thereof or PRP derived therefrom can be administered locally (e.g., osteoarthritis knee), parenterally, i.e., infusion, subcutaneous, intramuscular, intravenous, intradermal, intrathecal, and epidural, or topically, optically, or by inhalation, or rectally, or vaginal, or sub-lingually.

Therapeutic compositions and formulation thereof comprising PLCs or derivatives thereof or lysates thereof or PRP derived therefrom or PLC derivatives or precursor cells making the PLCs or derivatives thereof or PRP derived therefrom, used in accordance with the present disclosure, are prepared for storage by mixing the PLCs or derivatives thereof or lysates thereof or PRP derived therefrom or PLCs or PLC derivatives or precursor cells making the PLCs or derivatives thereof or PRP derived therefrom, having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN®, PLURONICS® or polyethylene glycol (PEG), Other pharmaceutically acceptable carriers can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.), water, salt solutions, alcohols, polyethylene glycols, gelatins, amyloses, magnesium stearates, talcs, silicic acids, viscous paraffins, hydroxymethylcelluloses, polyvinylpyrrolidones and the like. Pharmaceutical formulations to be used for in vivo administration are generally sterile. This is readily accomplished by filtration through sterile filtration membranes.

The formulations herein may also contain more than one active compound as necessary for the particular indication being treated, preferably those with complementary activities that do not adversely affect each other. For example, in addition to PLCs or derivatives thereof or lysates thereof or PRP derived therefrom or the PLCs or derivatives thereof or precursor cells making the PLCs or derivatives thereof or PRP derived therefrom, it may be desirable to include in the one formulation, an additional injury healing agent, e.g., an anti-inflammatory agent or an opioid drug. Alternatively, or additionally, the composition may further comprise a cytotoxic agent, cytokine, growth inhibitory agent, anti-hormonal agent, and/or cardioprotectant. Such molecules are suitably present in combination in amounts that are effective for the purpose intended.

The active ingredients (i.e., PLCs or derivatives thereof or lysates thereof or PRP derived therefrom) may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. Ed. (1980).

The active ingredients (i.e., PLCs or derivatives thereof or lysates thereof or PRP derived therefrom) may also be delivered in bioactive scaffolds made from natural (e.g., protein based) or synthetic (polymer or ceramic based) biomaterials. The bioactive scaffolds, in addition to the PLCs or derivatives thereof or lysates thereof or the PRP derived therefrom may optionally contain growth factors and other bioactive molecules. Among these are epidermal growth factor. TGF-alpha, TGF, beta, fibroblast growth factor, platelet derived growth factor, vascular endothelial growth factor, insulin-like growth factor, keratinocyte growth factor, and bone morphogenic protein to name a few. Growth factors may also be introduced into the scaffolds before or after the introduction of the PLCs or derivatives thereof or lysates thereof or the PRP derived therefrom.

Sustained-release preparations may be prepared. Suitable examples of sustained-release preparations include semi-permeable matrices of solid hydrophobic polymers containing the PLCs or derivatives thereof or lysates thereof or PRP derived therefrom or PLCs or PLC derivatives or precursor cells making the PLCs or derivatives thereof or PRP derived therefrom, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid. Biocompatible materials that may be present in a hydrogel include, e.g., permeable configurations or morphologies, such as polyvinyl alcohol, polyvinylpyrrolidone and polyacrylamide, polyethylene oxide, poly(-hydroxyethyl methacrylate); natural polymers such as polysaccharides, gums and starches; and include poly [alpha (4-aminobutyl)]-1-glycolic acid, polyethylene oxide, polyorthoesters, silk-elastin-like polymers, alginate, EVAc (poly(ethylene-co-vinyl acetate), microspheres such as poly (D, L-lactide-co-glycolide) copolymer and poly (L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, or agarose. In one embodiment, the hydrogel includes poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof. In some embodiments, the hydrogel includes collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxideypoly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS). Following polymers may be employed, e.g., natural polymers such as alginate, agarose, starch, fibrin, collagen, gelatin, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone. While polymers such as ethylene-vinyl acetate and lactic acid-glycolic acid enable release of molecules for over 100 days, certain hydrogels release proteins for shorter time periods. When encapsulated PLCs or derivatives thereof or lysates thereof or PRP derived therefrom or PLCs or PLC derivatives or precursor cells making the PLCs or derivatives thereof or PRP derived therefrom remain in the body for a long time, they may denature or aggregate as a result of exposure to moisture at 37 degree C., resulting in a loss of biological activity and possible changes in activity.

PLCs or derivatives thereof or lysates thereof or PRP derived therefrom or PLCs or PLC derivatives or precursor cells making the PLCs or derivatives thereof or PRP derived therefrom may be formulated in any suitable form for delivery to a target cell/tissue. For example, PLCs or derivatives thereof or lysates thereof or PRP derived therefrom or PLCs or PLC derivatives or precursor cells making the PLCs or derivatives thereof or PRP derived therefrom may be formulated as immunoliposomes. A “liposome” is a small vesicle composed of various types of lipids, phospholipids and/or surfactant which is useful for delivery of a drug to a mammal. The components of the liposome are commonly arranged in a bilayer formation, like the lipid arrangement of biological membranes. Liposomes containing the PLCs or derivatives thereof or lysates thereof or PRP derived therefrom or PLCs or PLC derivatives are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Another therapeutic agent is optionally contained within the liposome. See Gabizon et al., J. National Cancer Inst. 81(19):1484 (1989).

The formulations to be used for in vivo administration must be sterile. This is readily accomplished by filtration through sterile filtration membranes.

For treating injuries (e.g., OA, regenerating bone tissue to influence the process of bone regeneration and repair) or to regenerate aging skin or hair, in one embodiment, the PLCs or derivatives thereof or lysates thereof or PRP derived therefrom or PLCs or PLC derivatives or precursor cells making the PLCs or derivatives thereof or PRP derived therefrom are administered via any of the routes disclosed herein. The dosage administered via any of these routes is in the range of about 0.1 microgram/m² to about 10,000 microgram/m² per dose, generally one dose per day or per week for a total of one, two, three or four doses or multiple doses depending on the need. Alternatively, the dosage range is of about 0.1 microgram/m² to about 1000 microgram/m², about 0.1 microgram/m² to about 800 microgram/m², about 0.1 microgram/m² to about 600 microgram/m², about 0.1 microgram/m2 to about 400 microgram/m², about 0.1 microgram/m² to about 500 microgram/m², about 0.1 microgram/m² to about 300 microgram/m², about 0.1 microgram/m² to about 200 microgram/m², and about 0.1 microgram/m² to about 200 microgram/m². The dose may be administered once per day, once per week, multiple times per week, less than once per day or more than once per day, 2-3 times per day, multiple times per month, once per day, once per week or once per month or intermittently on a daily, weekly or a monthly basis (e.g., one or more than one dose is administered, daily, weekly, biweekly, triweekly or monthly) to relieve or alleviate symptoms of the injury or aging skin or disease related to the injury or aging skin. Administration may continue at any of the disclosed intervals until remission of the injury or aging of skin or symptoms related to the disease are being treated. Administration may continue after remission or relief of symptoms is achieved where such remission or relief is prolonged by such continued administration.

Emulsifying agents can natural or synthetic and include, but are not limited to, Cationic, e.g., benzalkonium chloride, benzethonium chloride. Anionic, e.g., alkali soaps (sodium or potassium oleate); amine soaps (triethanolamine stearate); detergents (sodium lauryl sulfate, sodium dioctyl sulfosuccinate, sodium docusate). Nonionic, e.g., sorbitan esters (Spans®), polyoxyethylene derivatives of sorbita esters (Tweens®), or glyceryl esters

Other agents that can be used as emulsifiers may include desoxycholic acid, diacetyl tartaric acid esters, egg yolk, glycerol, Gums, Irish Moss (carrageenan), Lecithin, Mono- and diglycerides, Monosodium phosphate, Monostearate, Ox bile extract, Propylene glycol, Soaps, Taurocholic acid (or its sodium salt).

In some embodiments of the present disclosure, PLCs or derivatives thereof or lysates thereof or the PRP derived therefrom or precursor cells making the PLCs or derivatives thereof or PRP derived therefrom are in a plasmalyte. Plasmalyte is a family of balanced crystalloid solutions. It closely mimics human plasma in its content of electrolytes, osmolality, and pH. These solutions also have additional buffer capacity and contain anions such as acetate, gluconate, and even lactate that are converted to bicarbonate, CO2, and water.

In some embodiments, MKs and platelets can also be derived from human induced pluripotent stem cells (iPSCs), hematopoietic stem cells, including but not limited, to CD34⁺ umbilical cord blood stem cells (UCB cells) (e.g., human CD34⁺ umbilical cord blood stem cells) such as those disclosed in a co-pending U.S. patent application Ser. No. 16/975,918, filed Jun. 25, 2020 and PCT Application based on U.S. Provisional application Ser. No. 63/025,209, filed May 15, 2020, incorporated herein in their entireties by reference, CD34⁺ mobilized peripheral blood cells (MPB cells) (e.g., CD34⁺ human mobilized peripheral blood), or CD34⁺ bone marrow cells. UCB cells are multipotent stem cells derived from blood that remains in the placenta and the attached umbilical cord after childbirth. MPB cells are multipotent stem cells derived from volunteers whose stem cells are mobilized into the bloodstream by administration of G-CSF or similar agent.

In some embodiments, MKs and platelets can be derived from other stem cell types, including but not limited to mesenchymal stem cells (MSC) (such as, adipose-derived mesenchymal stem cells (AdMSC)) or mesenchymal stem from other sources.

AdMSCs are derived from white adipose tissue, which is derived from the mesoderm during embryonic development and is present in every mammalian species, located throughout the body. Due to their wide availability and ability to differentiate into other tissue types of the mesoderm-including bone, cartilage, muscle, and adipose-ASCs may serve a wide variety of applications.

In the present disclosure, the stem cell cultures can be maintained independently of embryonic fibroblast feeder cells and/or animal serum. In some embodiments, serum-free, feeder-cell free alternatives can be utilized in the instant methods.

### Extracellular Vesicles (EVs)

In some embodiments, the present disclosure comprises microvesicles and exosomes (collectively referred to as extracellular vesicles (EVs)) or derivatives thereof, which are produced admixtures of the PLCs. Given that EVs or derivatives thereof carry growth factors, receptors, bioactive lipids, nucleic acids, such as mRNA and microRNA (miRNA) or siRNA, proteins, they are able to deliver important payloads to recipient cells (e.g., an osteoarthritis knee or to skin or a damaged organ or tissue or for use in regenerative medicine) making it even a richer resource to complement the PLCs with additional growth factors or other molecules for treating or ameliorating diseases, disorders, or injuries related to dry eye, osteoarthritis, tendon, ligament, bone repair, wound healing or wound-healing related disorders, alopecia or in skin rejuvenation or regeneration, for example.

EVs or derivatives thereof of the present disclosure can be isolated and purified, essentially separating them from an admixture comprising the PLCs of the present disclosure. Isolated or purified extracellular vesicles (EVs) or derivatives thereof, because of their ability to extensively travel throughout the body, can exert remarkable therapeutic effects when administered to a patient on their own. EVs have a fundamental immunomodulatory potential for treating or inhibiting diseases or disorders, or injuries related to dry eye, osteoarthritis, tendon, ligament, bone repair, wound healing or wound-healing related disorders, alopecia or in skin rejuvenation or regeneration. EVs could also be used as drug delivery system; they are able to cross biological barriers, including the blood-brain barrier and synovial membrane.

Advantageously, EVs or derivatives thereof can be internalized by recipient cells following receptor-ligand interactions and the varied assortment of bioactive molecules, derived from the cell of origin, such as proteins, bioactive lipids, and nucleic acids, can be transferred along with the proteins expressed on the EV surface.

In some embodiments, EVs or derivatives thereof may directly activate a recipient cell (e.g., donor platelets) by acting as signaling complexes. For example, EVs or derivatives thereof may bind to platelets by means of the P-selectin glycoprotein ligand-1 expressed on their surface and EVs or derivatives thereof from neutrophils expressing Mac-1 may induce donor platelet activation in a patient in need thereof. Such activations are advantageous because they facilitate activation of exogenous mechanisms that can enhance treatments related to the treating or ameliorating diseases, disorders, or injuries related to dry eye, osteoarthritis, tendon, ligament, bone repair, wound healing or wound-healing related disorders, alopecia or in skin rejuvenation or regeneration.

Compositions and methods comprising the extracellular vesicles (EV) or derivatives thereof of the present disclosure can be used in several therapies or co-therapies, such as for treating or ameliorating diseases, disorders, or injuries related to dry eye, osteoarthritis, tendon, ligament, bone repair, wound healing or wound-healing related disorders, alopecia or in skin rejuvenation or regeneration, delivery of genes, proteins or peptides, nucleic acids for the use in cellular or gene therapies, for example using vectors, e.g. adenovirus, lentivirus, to obtain novel microvesicles or exosomal gene (e.g., for gene therapy), peptide (for growth factors) or nucleic acid (e.g., siRNA or microRNA) delivery vehicles. Packaging within extracellular vesicles (EV) provides several advantages such as shielding the molecules from adverse cellular event that may neutralize the naked gene. Engineered extracellular vesicles (EV) could be used to carry drugs to specific sites of tissue damage, including but not limited to dry eye, osteoarthritis, tendon, ligament, bone repair, wound healing or wound-healing related disorders, alopecia or in skin rejuvenation or regeneration.

In some embodiments, the isolated extracellular vesicles (EV) derivatives thereof may then be stored until use by freezing at very low temperature, e.g., at −80° C. in presence of cryopreserving agents, such dimethylsulphoxide (DMSO) and glycerol used at optimal concentrations.

In some embodiments, an average diameter of extracellular vesicles (EV) derived from a population of iPSC derived platelets is less than 50% the diameter of the extracellular vesicles (EV) derived from a population of donor derived platelets having about the same number of platelets as the population of iPSC derived platelets. In some embodiments, the megakaryocyte or platelet is genetically modified to comprise a nucleic acid molecule encoding a therapeutic agent.

Extracellular vesicles (EV) are subcellular sized particles consisting of a membrane lipid bilayer and cellular content. Extracellular vesicles (EV) isolated or purified from an admixture comprising PLCs may exert both anti-inflammatory and pro-inflammatory function and have potential as vehicles for drug delivery.

In some embodiments, the diameter of the instant extracellular vesicles (EV) is 0.1 and 4 μm. In some embodiments, the diameter of the instant extracellular vesicles (EV) is 0.1 and 3 μm. In some embodiments, the diameter of the instant extracellular vesicles (EV) is 0.1 and 2.5 μm. In some embodiments, the diameter of the instant extracellular vesicles (EV) is 0.1 and 2 μm. In some embodiments, the diameter of the instant extracellular vesicles (EV) is 0.1 and 1.5 μm. In some embodiments, the diameter of the instant extracellular vesicles (EV) is 0.1 and 1.0 μm. In some embodiments, the diameter of the instant extracellular vesicles (EV) is 0.1 and 0.9 μm. In some embodiments, the diameter of the instant extracellular vesicles (EV) is 0.1 and 0.8 μm. In some embodiments, the diameter of the instant extracellular vesicles (EV) is 0.1 and 0.7 μm. In some embodiments, the diameter of the instant extracellular vesicles (EV) is 0.1 and 0.6 μm. In some embodiments, the diameter of the instant extracellular vesicles (EV) is 0.1 and 0.5 μm. In some embodiments, the diameter of the instant extracellular vesicles (EV) is 0.1 and 0.4 μm. In some embodiments, the diameter of the instant extracellular vesicles (EV) is 0.1 and 0.3 μm. In some embodiments, the diameter of the instant extracellular vesicles (EV) is 0.1 and 0.2 μm. In some embodiments, the diameter of the instant extracellular vesicles (EV) is 0.2 and 1 μm. In some embodiments, the diameter of the instant extracellular vesicles (EV) is 0.3 and 1 μm. In some embodiments, the diameter of the instant extracellular vesicles (EV) is 0.4 and 1 μm. In some embodiments, the diameter of the instant extracellular vesicles (EV) is 0.5 and 1 μm. In some embodiments, the diameter of the instant extracellular vesicles (EV) is 0.6 and 1 μm. In some embodiments, the diameter of the instant extracellular vesicles (EV) is 0.7 and 1 μm. In some embodiments, the diameter of the instant extracellular vesicles (EV) is 0.8 and 1 μm. In some embodiments, the diameter of the instant extracellular vesicles (EV) is 0.9 and 1 μm. In some embodiments, the diameter of the instant extracellular vesicles (EV) is 0.2 and 2 μm. In some embodiments, the diameter of the instant extracellular vesicles (EV) is 0.3 and 2 μm. In some embodiments, the diameter of the instant extracellular vesicles (EV) is 0.4 and 2 μm. In some embodiments, the diameter of the instant extracellular vesicles (EV) is 0.5 and 2 μm. In some embodiments, the diameter of the instant extracellular vesicles (EV) is 0.6 and 2 μm. In some embodiments, the diameter of the instant extracellular vesicles (EV) is 0.7 and 2 μm. In some embodiments, the diameter of the instant extracellular vesicles (EV) is 0.8 and 2 μm. In some embodiments, the diameter of the instant extracellular vesicles (EV) is 0.9 and 2 μm. In some embodiments, the diameter of the instant extracellular vesicles (EV) is 1.0 and 2 μm. In some embodiments, the diameter of the instant extracellular vesicles (EV) is 1.5 and 2 μm. In some embodiments, the diameter of the instant extracellular vesicles (EV) is 2.0 and 2.5 μm.

Extracellular vesicles (EV) derivatives thereof may be conjugated to one or more cytotoxic agents by mechanisms disclosed in the foregoing. One or more cytotoxic agents may be imbibed into the extracellular vesicles (EV) derivatives thereof by mechanisms also disclosed in the foregoing. Cytotoxic agents are also disclosed in the foregoing. Diseases and disorders that can be cured or mitigated by the use of EVs derivatives thereof alone or in combination with the PLCs or derivatives thereof of the present disclosure are also disclosed below.

In some embodiments, EVs, whether modified or not (e.g., bioengineered or conjugated) may be developed for therapeutic use independent of the PLCs or derivatives therefrom. For example, a patient in need of a treatment predominantly involving microvesicles or derivatives thereof will be administered microvesicle-based treatment or exosome-based treatment or a combination of both. For example, MVs or exosomes incorporated with exogenous growth factors, cytokines, or siRNAs can be used for efficient silencing of a target MAPK gene in monocytes and lymphocytes or deliver growth factors siRNAs (e.g., VEGF-siRNA) targeting, for example, regenerating bone tissue to influence the process of bone regeneration and repair and alopecia. Advantageously, MVs could be used as more efficient delivery vehicles to direct specific targeting of novel therapeutics without immunogenicity and adverse effects.

In some embodiments, the EV-based treatment may be administered prior to PLC-based treatment. In some embodiments, PLC-based treatment may be administered prior to EV-based treatment. In some embodiments, PLCs and EVs are administered as admixtures. Also contemplated are treatments in which admixtures comprising PLCs and EVs are administered followed by treatment regiments comprising essentially of EV or derivatives thereof or comprising essentially of PLCs or derivatives thereof-based treatment depending on a patient's need.

Some embodiments of the present disclosure provide a composition suitable for cryopreservation including the PLCs or derivatives thereof or lysates thereof or PRP derived therefrom or precursor cells making the PLCs or derivatives thereof or PRP derived therefrom and a cryopreservation agent. In some embodiments, the cryopreservation agent is a sugar, alcohol, polymer, protein, or a combination thereof. In some embodiments, the cryopreservation agent is DMSO, glycerol, trehalose, cellulose, or a combination thereof. In some embodiments, the cryopreservation agent is DMSO. In some embodiments, the cryopreservation agent is glycerol or arabinogalactan. In some embodiments, the cryopreservation agent is DMSO and glycerol. In some embodiments, the cryopreservation agent is trehalose or propylene glycol or albumin or a combination thereof. In some embodiments, the cryopreservation agent is cellulose. A combination comprising one or more of the cryopreservation agents is also encompassed in the present disclosure. In some embodiments, the composition is frozen, i.e., the composition is stored at a temperature between about −80° C. and −200° C. Cryoprotective agents can be classified as penetrating and non-penetrating. Non-penetrating cryoprotective agents alter only the freezing characteristics of the extracellular medium whereas penetrating cryoprotective agents can modify both the intracellular and the extracellular medium composition.

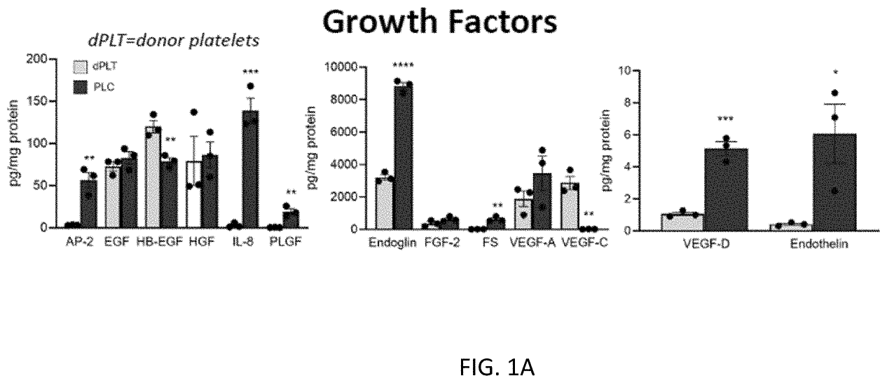

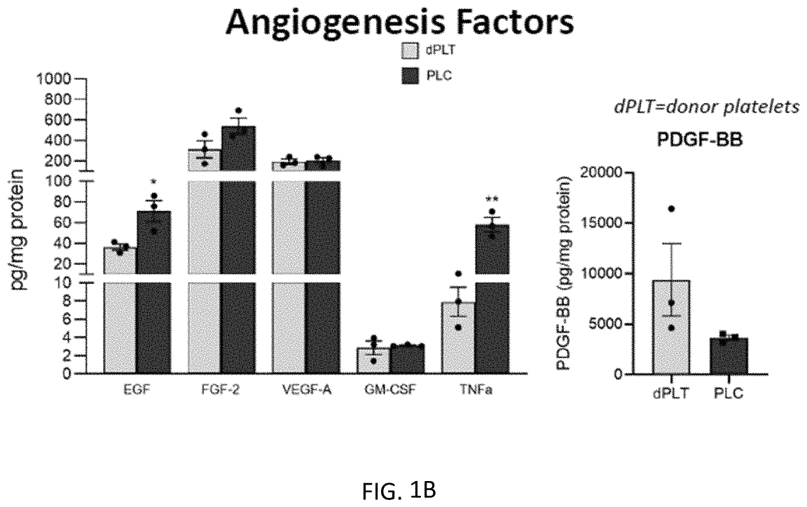

The PLCs or derivatives thereof or lysates thereof or PRP derived therefrom or precursor cells making the PLCs or derivatives thereof or PRP derived therefrom, in a non-limiting embodiment, are rich in growth factors or could be supplemented with growth factors, such as but not limited to Angiopoitin-1, bFGF, EGF, FGF, HGF, IGF-I, IGF-II, PDAF, PDEGF, PDGF AA and BB, TGF-beta 1, 2, and 3, and VEGF, cytokines (e.g., IL-1B, IL-6, IL-8, IL-10, TNF-A, MIG, MCP-1, IP-10) or chemokines (e.g., ENA-78 (CXCL5), IL-8 (CXCL8), monocyte chemotactic protein, MCP-3 (CCL7), MIP-1A (CCL3), NAP-2 (CXCL7), PF4 (CXCL4), or inflammatory mediators (e.g., PGE2), macrophage inflammatory protein-1 (MIP-1) or regulated on activation, normal T cell expressed and secreted (RANTES), their concentrations often higher than that obtained from donor platelets. The concentrations of the growth factors or cytokines in PRP enriched from PLCs or derivatives thereof or lysates thereof or PRP derived therefrom or PLCs or derivatives thereof (e.g., lysates form PLCs or derivatives therefrom) or precursor cells making the PLCs or derivatives thereof or PRP derived therefrom may be diluted as desired by a subject's need. In some embodiments, PLCs can be genetically engineered to express one or more of the growth factors discussed herein. Non-limiting examples of endogenous PLC-based growth factors are shown in FIGS. 1A and 1B.

PLCs or derivatives thereof or lysates thereof or PRP derived therefrom or precursor cells making the PLCs or derivatives thereof or PRP derived therefrom or a composition thereof can be administered or applied to damaged tissues of the musculoskeletal system, such as sprained ligaments (which attach bone to bone), strained or torn muscles can be torn, tendon ruptures, osteoarthritis, fractured bones can be cracked or broken or the surrounding injured tissues, bone dislocations, i.e., where the bones in a joint may become completely separated from each other (called dislocation) or only partly out of position (called subluxation), sprains, strains, or other musculoskeletal injuries, completely or partially torn tendons, muscle spasms, nerve pains, whiplash, athletic injuries, myocardial infarction or ischemia-related disorders (e.g., limb ischemia, lower extremity ischemia, myocardial ischemia, organ ischemia, ischemic heart disease or the like), surgical procedures, which are difficult to heal, lung diseases, cardiac diseases,

The site of a tissue damage or injury may be determined by well-established techniques, such as but not limited to, imaging studies which include, but are not limited, to MRI, X-ray, CT scan, X-rays which may be done to check for fractures and dislocations, which may also be present. In addition, x-rays can show abnormalities in the position of bones that may suggest a sprain or other soft-tissue injury. Magnetic resonance imaging (MRI), MRI can show soft tissues, which are not usually visible on x-rays. MRI thus helps detect injury to tendons, ligaments, cartilage, and muscle. Other techniques, such as but not limited to, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging NOGA mapping and ultrasound techniques may also be applied to determine a site of injury. A physician may consult a subject before one or more of the above testing is performed.

The PLCs or derivatives thereof or lysates thereof or PRP derived therefrom or precursor cells making the PLCs or derivatives thereof or PRP derived therefrom or a composition thereof may be administered through different routes, such as but not limited to, systemic, topical, with the use of an implantable device, such as but not limited to stents, mesh (e.g., a polymer mesh or a bioabsorbable mesh), adhesive biomaterials (e.g., naturally derived or synthetic biopolymers) or other devices know to one of skill in the art. For example, variables such as proper timing, treatment periodicity, location and technique for knee injection (e.g., an intravenous or intradermal injection) in patients suffering from knee osteoarthritis treatment may vary from a single or multiple PLC applications. PLCs or derivatives therefrom can be applied on a daily, weekly (3-weekly PLC injections) or monthly basis (two monthly injections, three injections at 15-day intervals or 21-day intervals. Treatment regimens may vary depending on the need of a patient). Location and technique for the application of the PLCs or derivatives thereof or lysates thereof or PRP derived therefrom (e.g., local injection to a knee) could be lateral, supero-lateral, para-patellar, and lateral mid-patellar, among others. In each such treatment, optionally one or more of other therapeutic agents can be co-administered simultaneously, or at periodic intervals from one another.

If stents are used, the stents could be closed cell or open cell stents, which are well known to one of skill in the art. PLCs or derivatives thereof or lysates thereof or PRP derived therefrom may be bonded directly to a metal stent or be bonded to a matrix polymer, which acts as a drug reservoir to ensure source retention during deployment and a uniform distribution on the stent. The types, compositions, and designs of the polymers coated on the stent generally will dictate the eluting kinetic of the sustain time release of the PLCs or derivatives thereof or lysates thereof or PRP derived therefrom over a period of weeks or months following the implantation in situ. The coating materials can be categorized as organic vs inorganic, bioerodable vs nonbioerodable, and synthetic vs naturally occurring substances.

In some embodiments, the composition comprising The PLCs or derivatives thereof or lysates thereof or PRP derived therefrom or precursor cells making the PLCs or derivatives thereof or PRP derived therefrom comprises between 1 and 25 wt. %, 25 and 50 wt. %, 50 and 75 wt. %, 75 and 100 wt. % of PLCs or derivatives thereof or platelet-rich plasma derived therefrom.

In some embodiments, the composition comprises between 1 and 25 wt. %, 25 and 50 wt. %, 50 and 75 wt. %, 75 and 98 wt. % of a bulking agent. In some embodiments, the composition comprises between 1 and 25 wt. %, 25 and 50 wt. %, 50 and 75 wt. %, 75 and 98 wt. % of at least one excipient or carrier. Once the wt. % of PLCs or derivatives thereof or platelet-rich plasma derived therefrom, bulking agent, excipient or carrier can be adjusted accordingly. For example, in an aspect of the present disclosure the composition may comprise a) between 1 and 50 wt. % of PLCs or derivatives thereof or lysates thereof or platelet-rich plasma derived therefrom, b) between 1 and 25 wt. % of a bulking agent, and/or c) between 50 and 98 wt. % of at least one excipient or carrier.

In some embodiments, the adhesive biopolymer materials may include, but are not limited to, polycarbophil (PCP), Xanthan gum, pectin, Hydroxypropyl methylcellulose (HPMC) or hypromellose Carbopol 1342P, Carbopol 974P, Chitosan, Carbopol 971P, hydroxypropylmethyl-cellulose (Methacel K100M), CMC-Na, hydroxypropylmethyl-cellulose (Methacel K15M), gelatin, Acacia gum or a combination thereof. When the PLCs or derivatives thereof or lysates thereof or PRP derived therefrom is contained in adhesive biomaterials, the adhesive bioagents adhere to the target sites in order to extend the retention time of the PLCs or derivatives thereof or lysates thereof or PRP derived therefrom in a lesion (e.g., ocular lesion), and improve the treatment effect of local disease (e.g., dry eye disease). Higher local drug concentration and the close contact with the site of absorption can not only promote absorption of the drug, but also increase concentration gradient. The adhesive biopolymers may modulate transport pathways by opening epithelial tight junctions to promote the diffusion of the PLCs or derivatives thereof or lysates thereof or PRP derived therefrom. In addition, PLCs or derivatives thereof or lysates thereof or PRP derived therefrom may adhere to the mucosa directly and is absorbed by the mucosal capillaries to increase the bioavailability. Furthermore, the use of adhesive biomaterials permit in the preparation of controlled release formulations for the PLCs or derivatives thereof or lysates thereof or PRP derived therefrom, which can reduce the frequency of administration to improve a patient's health in need of PLC-based therapy.

In some embodiments, the PLCs or derivatives thereof or lysates thereof or PRP derived therefrom or precursor cells making the PLCs or derivatives thereof or PRP derived therefrom maybe applied to dry eye disease. Dry eye diseases fall into two categories, which are (i) tear-deficient and (ii) evaporative. Sjogren's syndrome or non-Sjogren's syndrome causes are inclusive of exocrinopathy in which lacrimal secretion deficiency occurs due to an autoimmune process that affects the lacrimal glands, salivary glands, and other organs of the body (Sjogren's syndrome) or inclusive of Non-Sjogren's syndrome caused by lacrimal diseases or lacrimal obstruction and by reflex alterations, without autoimmune factor role. Some causes of Sjogren's syndrome or non-Sjogren's syndrome are age-related dry eye, congenital alacrima, familial dysautonomia, sarcoidosis, lymphoma, AIDS (acquired immunodeficiency syndrome), gland denervation, lacrimal obstruction as in pemphigus, trigeminal injury, diabetes, neurotrophic keratopathy, use of contact lenses, and motor reflex block due to VII pair injury. The evaporative causes of dry eye disease are due to oil deficit, lid changes, use of contact lenses, or ocular surface diseases, as allergic conjunctivitis, and some of the iatrogenic dry eye that occurs after the use of systemic or topical medications or after surgeries or nonsurgical procedures.

The PLCs or derivatives thereof or lysates thereof PRP derived therefrom or precursor cells making the PLCs or derivatives thereof or PRP derived therefrom may be applied in eyedrops. For example, two to three milliliters of this concentrate may be placed in sterile eyedrops. The eyedrops can kept at −20° C. for long term storage. When the use of the PLCs or derivatives thereof or lysates thereof or PRP derived therefrom-containing eye drop is desired, a patient may thaw the eye drops and keep it at +4° C. The patients may use these eyedrops one to six times a day for 1 month, 2 months, 3 months or more. In some embodiments, the patient may use the eye drops once a day for 1 weak, 2 weeks, 3 weeks, or four weeks or more.

In some embodiments, the PLCs or derivatives thereof or lysates thereof or PRP derived therefrom or precursor cells making the PLCs or derivatives thereof or PRP derived therefrom may be applied via an injection or topically to areas of skin such as aging skins in the face, scalp, neck, chest, hands, arms, legs, abdomen, or buttocks. It is envisioned that the PLCs or derivatives thereof or lysates thereof or PRP derived therefrom or precursor cells making the PLCs or derivatives thereof or PRP derived therefrom maybe applied to scalp to prolong the life of hair follicles and increase hair growth. For use on skin or the scalp, PLCs or derivatives thereof or lysates thereof or PRP derived therefrom or the precursor cells making the PLCs or derivatives thereof or PRP derived therefrom are provided in a composition comprising cosmetically acceptable carrier. For example, in an aspect of the present disclosure is provided a method for generating a hair follicle in a scalp or hair-loss region thereof of a subject comprising contacting the scalp or a hair-loss region therein with the PLCs or derivatives thereof or lysates thereof or PRP derived therefrom or precursor cells making the PLCs or derivatives thereof or PRP derived therefrom. Another precursor cell that is capable of differentiation into a hair follicle cell may also be included therewith such that the precursor cell is an inductive cell that is capable of inducing differentiation of an uncommitted epidermal cell into a hair follicle cell when combined with the PLCs or derivatives thereof or lysates thereof or PRP derived therefrom or the precursor cells making the PLCs or derivatives thereof of the present disclosure. In some embodiments, PLCs or derivatives thereof or lysates thereof or PRP derived therefrom or precursor cells making the PLCs or derivatives thereof or PRP derived therefrom (i.e., sources for the PRP) is applied first, followed by an inductive cell, or the PRP or the source thereof and the inductive cell are applied simultaneously, or the inductive cell is applied first followed by PRP or the PRP source application. Except when applied simultaneously, one component may be applied within few hours, few days or few weeks of the other depending on the desired application needs of a subject.

Second therapeutic agents for use with the PLCs or derivatives thereof or lysates thereof or PRP derived therefrom could also be selected from one or more of nonsteroidal anti-inflammatory drugs (NSAIDs), cox 2 inhibitors, Suitable corticosteroids include 11′-alpha, 17-alpha, 21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha, 21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxy cortisone; 18-oxocortisol; 21-acetoxypregnenolone; 21-deoxy aldosterone; 21-deoxycortisone; 2-deoxyecdysone; 2-methylcortisone; 3-dehydroecdysone; 4-pregnene-17-alpha, 20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin. Other compounds that may be used as a substitute for or in addition to a corticosteroid in the methods, compositions, and kits of the present disclosure include, without limitation, A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495.times.(GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

It may be desirable to administer to the patient other compounds, such as a corticosteroid, tetra-substituted pyrimidopyrimidine, glucocorticoid, beta-catenin protein or polypeptide or agonist thereof, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), glucocorticoid receptor modulator, or DMARD. Combination therapies of the present disclosure are especially useful for the treatment of immunoinflammatory disorders in combination with other agents—either biologics or small molecules—that modulate the immune response to positively affect disease. Such agents include those that deplete key inflammatory cells, influence cell adhesion, or influence cytokines involved in immune response. This last category includes both agents that mimic or increase the action of anti-inflammatory cytokines such as IL-10, as well as agents inhibit the activity of pro-inflammatory cytokines such as IL-6, IL-1, IL-2, IL-12, IL-15 or TNF alpha. Agents that inhibit TNF alpha include etanercept, adelimumab, infliximab, and CDP-870. In this example (that of agents blocking the effect of TNF alpha), the combination therapy reduces the production of cytokines, etanercept or infliximab act on the remaining fraction of inflammatory cytokines, providing enhanced treatment. Small molecule immunomodulators include, e.g., p38 MAP kinase inhibitors such as VX 702, SCIO 469, doramapimod, RO 30201195, SCIO 323, TACE inhibitors such as DPC 333, ICE inhibitors such as pranalcasan, and IMPDH inhibitors such as mycophenolate and merimepodib.

Other therapeutic agents that can be administered, prior to, at the same time or after a gap, with the PLCs or derivatives thereof lysates thereof or PRP derived therefrom include, thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL™); beta-lapachone; lapachol; colchicines; betulinic acid; a camptothecin (including the synthetic analogue topotecan (HYCAMTIN™), CPT-11 (irinotecan, CAMPTOSAR™), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gammal I and calicheamicin omegall; CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN™, morpholino-doxorubicin, cyanomorpholino-doxorubicin, 2-pyrrolino-doxorubicin, doxorubicin HCl liposome injection (DOXIL™), liposomal doxorubicin TLC D-99 (MYOCET™), peglylated liposomal doxorubicin (CAELYX™), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR™), tegafur (UFTORAL™), capecitabine (XELODA™), an epothilone, and 5-fluorouracil (5-FU); combretastatin; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK™ polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE™, FILDESIN™); dacarbazine; mannomustine; mitobronitol; mitolactol; pipobroman; gacytosine; arabinoside (“Ara-C”); thiotepa; taxoid, e.g., paclitaxel (TAXOL™, Bristol-Myers Squibb Oncology, Princeton, N.J.), albumin-engineered nanoparticle formulation of paclitaxel (ABRAXANE™), and docetaxel (TAXOTERE™, Rhome-Poulene Rorer, Antony, France); chloranbucil; 6-thioguanine; mercaptopurine; methotrexate; platinum agents such as cisplatin, oxaliplatin (e.g., ELOXATIN™), and carboplatin; vincas, which prevent tubulin polymerization from forming microtubules, including vinblastine (VELBAN™), vincristine (ONCOVIN™), vindesine (ELDISINE™, FILDESIN™), and vinorelbine (NAVELBINE™); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN™); bisphosphonates such as clodronate (for example, BONEFOS™ or OSTAC™), etidronate (DIDROCAL™), NE-58095, zoledronic acid/zoledronate (ZOMETA™), alendronate (FOSAMAX™), pamidronate (AREDIA™), tiludronate (SKELID™), or risedronate (ACTONEL™); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R) (e.g., erlotinib (Tarceva™)); and VEGF-A that reduce cell proliferation; vaccines such as THERATOPE™ vaccine and gene therapy vaccines, for example, ALLOVECTIN™ vaccine, LEUVECTIN™ vaccine, and VAXID™ vaccine; topoisomerase 1 inhibitor (e.g., LURTOTECAN™); rmRH (e.g., ABARELIX™); BAY439006 (sorafenib; Bayer); SU-11248 (sunitinib, SUTENT™, Pfizer); perifosine, COX-2 inhibitor (e.g., celecoxib or etoricoxib), proteosome inhibitor (e.g., PS341); bortezomib (VELCADE™); CCI-779; tipifarnib (R11577); orafenib, ABT510; Bcl-2 inhibitor such as oblimersen sodium (GENASENSE™); pixantrone; EGFR inhibitors; tyrosine kinase inhibitors; serine-threonine kinase inhibitors such as rapamycin (sirolimus, RAPAMUNE™); farnesyltransferase inhibitors such as lonafarnib (SCH 6636, SARASAR); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin, and pharmaceutically acceptable salts, acids or derivatives of any of the above; as well as combinations of two or more of the above.

Therapeutic agents as defined herein also include “anti-hormonal agents” or “endocrine therapeutics” which act to regulate, reduce, block, or inhibit the effects of hormones that can promote the growth of cancer. They may be hormones themselves, including, but not limited to: anti-estrogens and selective estrogen receptor modulators (SERMs), including, for example, tamoxifen (including NOLVADEX™ tamoxifen), raloxifene, droloxifene, 4-hydroxytamoxifen, trioxifene, keoxifene, LY117018, onapristone, toremifene; aromatase inhibitors that inhibit the enzyme aromatase, which regulates estrogen production in the adrenal glands, such as, for example, 4(5)-imidazoles, aminoglutethimide, MEGASE™ megestrol acetate, AROMASIN™ exemestane, formestanie, fadrozole, RIVISOR™ vorozole, FEMARA™ letrozole, and ARIMIDEX™ anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME™ ribozyme) and a HER2 expression inhibitor; vaccines such as gene therapy vaccines, for example, ALLOVECTIN™ vaccine, LEUVECTIN™ vaccine, and VAXID™ vaccine; PROLEUKIN™ rlL-2; LURTOTECAN™ topoisomerase 1 inhibitor; ABARELIX™ rmRH; Vinorelbine and Esperamicins, and pharmaceutically acceptable salts, acids or derivatives of any of the above; as well as combinations of two or more of the above.

### Immunomodulatory Drug

In some embodiments, the present disclosure also encompasses immunomodulatory drugs for use with the PLCs or derivatives thereof or lysates thereof or precursor cells making the PLCs or derivatives thereof lysates thereof or PRP derived therefrom of the present disclosure. The term “immunomodulatory drug” refers to a class of drugs that modifies the immune system response or the functioning of the immune system, such as by the stimulation of antibody formation and/or the inhibition of peripheral blood cell activity, and include, but are not limited to, thalidomide (a-N-phthalimido-glutarimide) and its analogues, REVLIMID™ (lenalidomide), ACTI-MID™ (pomalidomide), OTEZLA™ (apremilast), and pharmaceutically acceptable salts or acids thereof.

Therapy, according to the present disclosure, may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Optionally, a person having a greater risk of developing an inflammatory disease (e.g., a person who is undergoing age-related hormonal changes) may receive treatment to inhibit or delay the onset of symptoms.

In combination therapy, the dosage and frequency of administration of each component of the combination can be controlled independently. For example, one compound may be administered once, twice, or three times per day, or week or month, while the second compound may be administered once per day, week or month. Combination therapy may be given in on-and-off cycles that include rest periods so that the patient's body has a chance to recover from any as yet unforeseen side effects. The compounds may also be formulated together such that one administration delivers both compounds. Except when administered simultaneously, one component in the combination therapy may be administered within few hours, few days or few weeks of the other depending on the desired administration needs of a subject.

In some embodiments, less than 10⁶ PLCs or about 10⁶ to 10⁷ PLCs or about 10⁷ to 10⁸ PLC or about 10⁸ to 10⁹ PLCs or about 10⁹ to 10¹⁰ PLCs or greater than 10¹⁰ PLCs are administered to the subject. For example it could be about (1, 2, 3, 4, 5, 6, 7, 8, or 9)×10⁶ to (1, 2, 3, 4, 5, 6, 7, 8, or 9)×10⁷ PLCs, about (1, 2, 3, 4, 5, 6, 7, 8, or 9)×10⁷ PLCs to (1, 2, 3, 4, 5, 6, 7, 8, or 9)×10⁸ PLCs, or about (1, 2, 3, 4, 5, 6, 7, 8, or 9)×10⁸ PLCs to (1, 2, 3, 4, 5, 6, 7, 8, or 9)×10⁹ PLCs or about (1, 2, 3, 4, 5, 6, 7, 8, or 9)×10⁹ PLCs to (1, 2, 3, 4, 5, 6, 7, 8, or 9)×10¹⁰ PLCs may be administered to the subject.

The present disclosure provides a kit comprising the PLCs or derivatives thereof or lysates thereof or PRP derived therefrom (i.e., lysates derived from PLCs or derivatives thereof) or precursor cells making the PLCs or derivatives thereof or PRP derived therefrom for use according to the present disclosure, and optionally carriers, buffers, emulsifiers or excipients, the kit further comprising instructions for administration to a subject. The kit may optionally contain additionally one or more drugs in one or more containers. The kit may further comprise a label or package insert. The package insert will provide instructions for use, dosage indications, administration, contraindications and/or warnings concerning the use of such contents of the kit. containers may include vials, bottles, syringes, blister pack, and the like. The active agents (e.g., PLCs or any additional drug contained in the kit) ordinarily can be stored as a solid composition, a lyophilized formulation or as an aqueous solution.

The following examples are put forth so as to provide those of ordinary skill in the art with a complete disclosure and description of how to make and use the assay, screening, and therapeutic methods of the disclosure, and are not intended to limit the scope of what the inventors regard as their disclosure.

### EXAMPLES

### Example 1: PLC Osteoarthritis (OA) Study in Rat MIA Model Showing Significant Improvement in OA Conditions

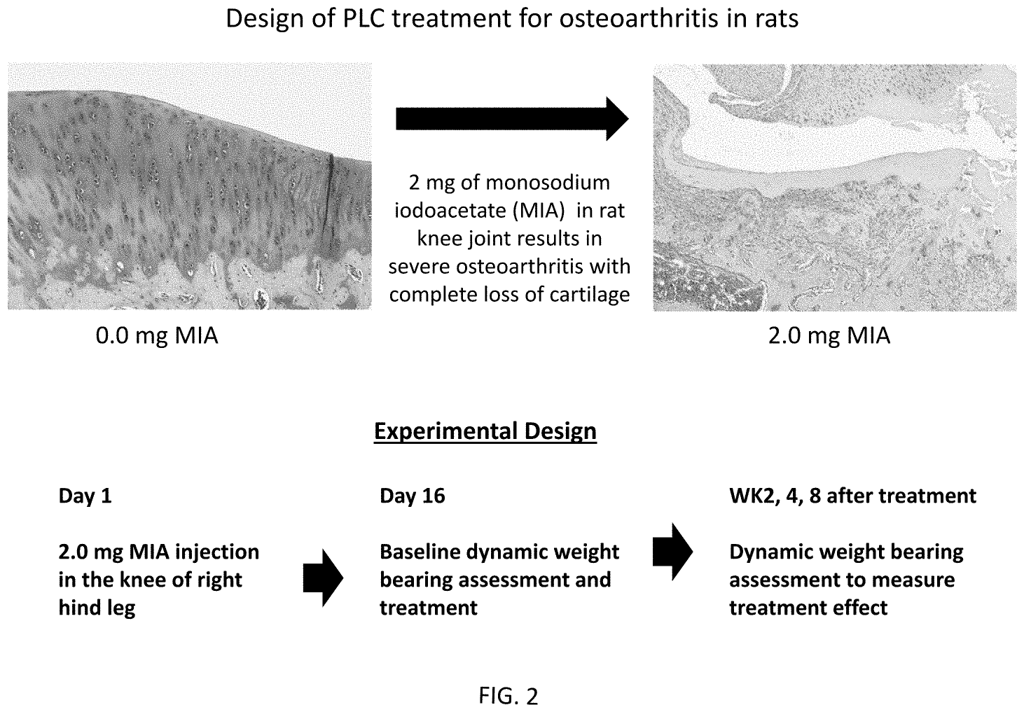

The rat monoiodoacetate (MIA) osteoarthritis model was used in this study as shown in FIG. 2. MIA inhibits glyceraldehyde-3-phosphate, causing chondrocyte cell death, leading to cartilage degeneration and subchondral bone pathology similar to human OA disease.

Groups of 20 male Sprague Dawley rats were given a 2 mg intra-articular injection of MIA into the right knee on day 0. On day 18, rats were treated with a single dose of plasmalyte buffer (negative control), PLCs, PLC lysate, or human PRP by intra-articular injection into the right knee. Oral dexamethasone was used as a positive control, but rats on this regimen began to lose weight so treatment was stopped early in the study.

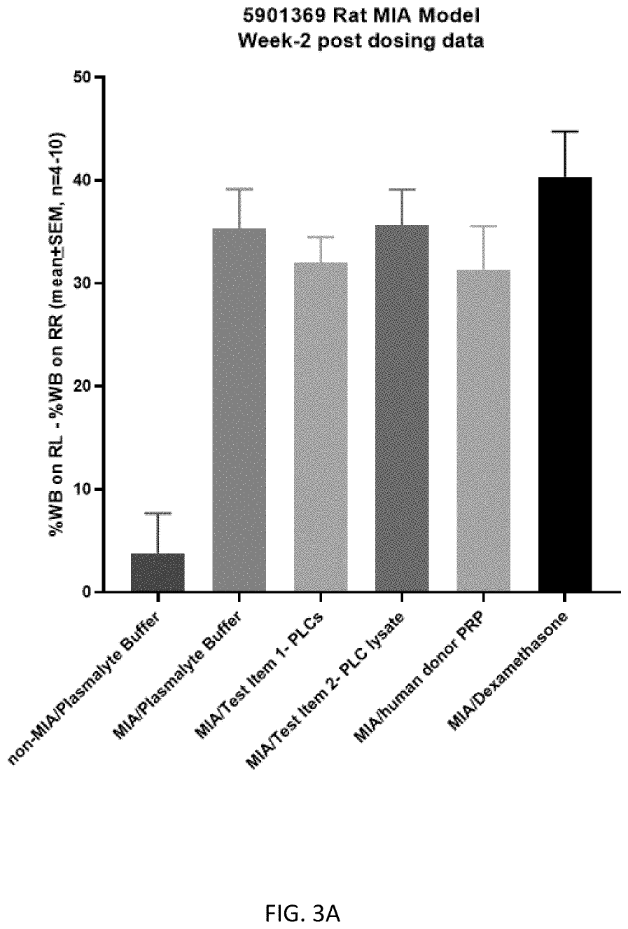

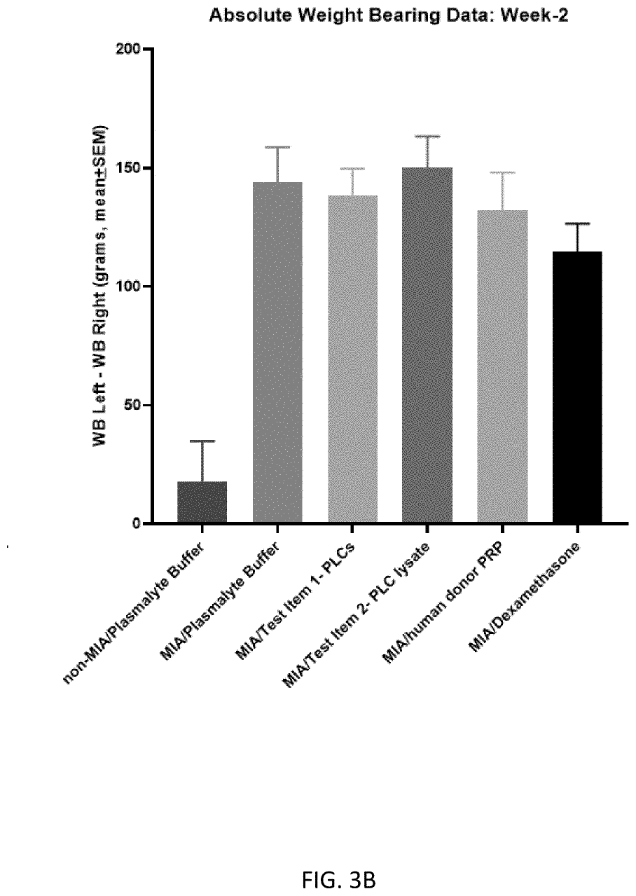

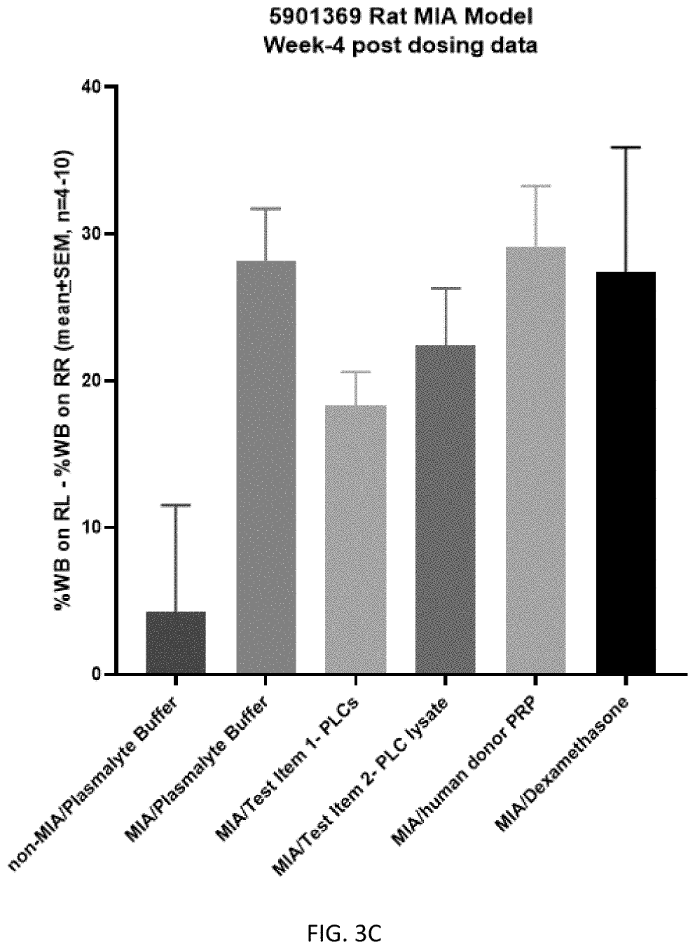

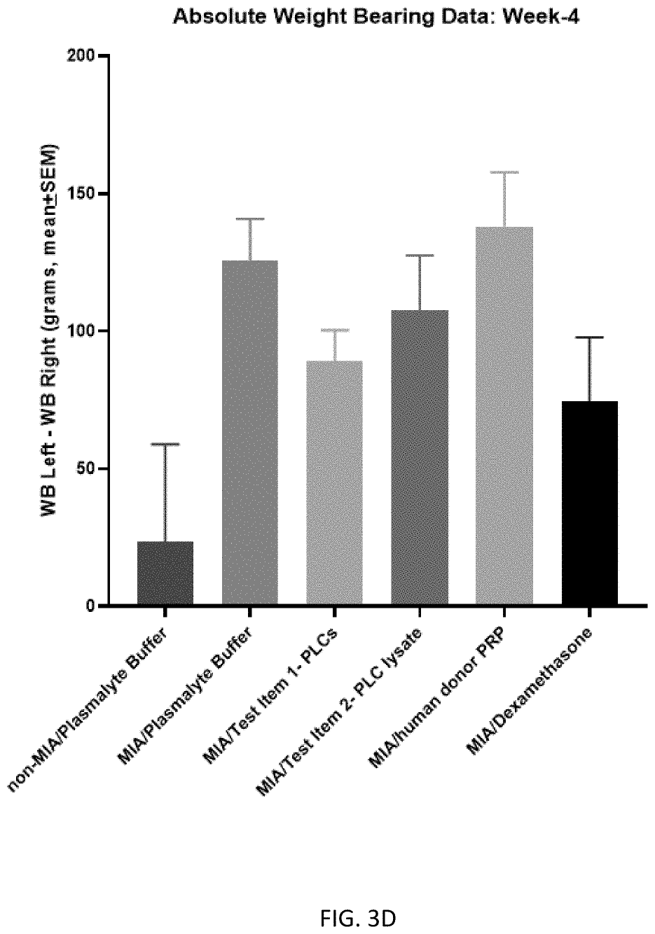

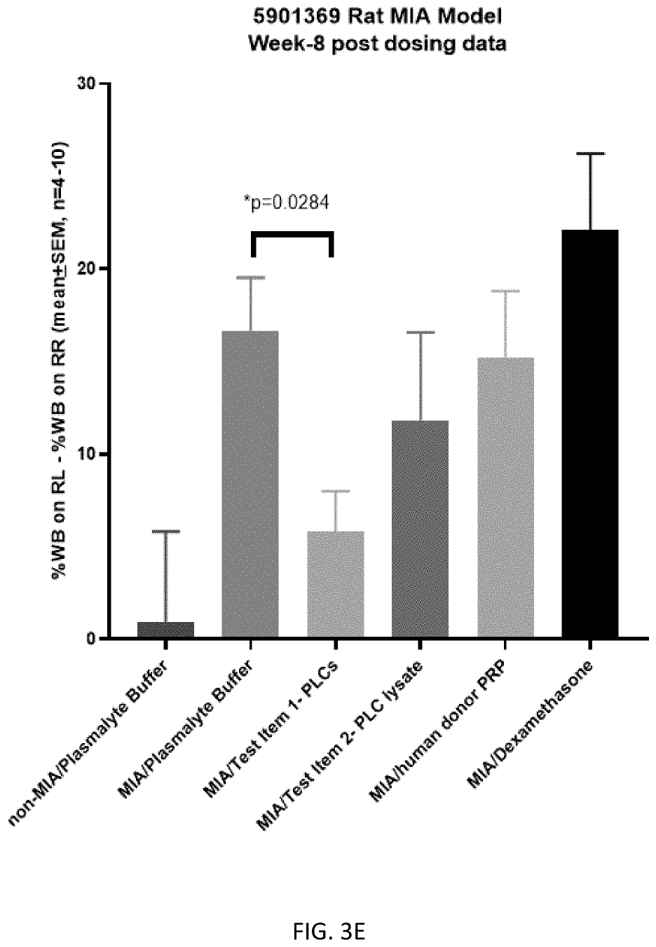

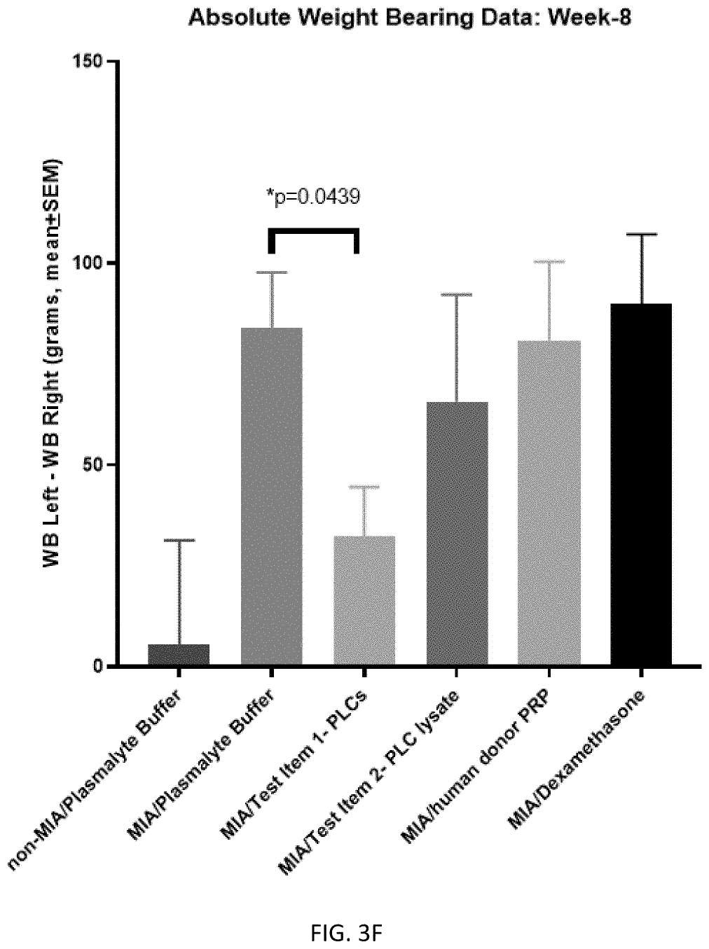

Dynamic Weight Bearing was used to assess pain in these animals. The rat stands with the rear left paw (RL) on one balance and the rear right paw (RR) on another balance. A healthy rat will place approximately equal weight on both legs while an osteoarthritic rat will put more weight on the healthy limb (RL) compared to the diseased one (RR). Successful treatment with PLCs resulted in pain cessation and lead to lowering the differential weight bearing over time. Weight bearing was tested at 2 (FIGS. 3A-3B), 4 (FIGS. 3C-3D) and 8 (FIGS. 3E-3F) weeks after therapeutic dosing. At 8 weeks, PLC-treated rats showed significant improvements to their OA condition. Successful treatment with PLCs resulted in pain cessation in the diseased limb and led to lowering the differential weight bearing over time.

### Example 2: PLC-Based Diabetic Wound Healing

Experimental Procedures.

Animals: Species: Rats; Strain: ZDF (ZDF-Leprfa/Crl, obese), Lean control rats; Source of Animals: Charles River Lab; Age or weight: 16 weeks of age; Sex: Male

Randomization: Baseline blood glucose and bodyweight will be measured. Animals will be distributed to treatment groups based upon these parameters. Rats with blood glucose >14 mmol/L or 252 mg/dl will used for the study.

Study Design: Study duration: 15 days; Number of Groups: 2; Number of animals per group: 10; Total number of animals: 20

Methods: Diabetes Confirmation: Male obese catheterized ZDF rats were obtained from the Charles River Lab. All rats were single-housed. Diabetes status was confirmed by assessing blood glucose from a tail snip. Blood glucose was measured using Glucocard Vital glucometers (Arkray, Minneapolis, Minn.) and levels reported as mg/dL. Glucometers were calibrated prior to each study. Blood (<5 μL) was acquired from a tail snip and directly applied to a glucose test strip. The rats with glucose levels not lower than 14 mmol/L will be applied to the study.

Wound induction, treatment and post-wound monitoring: the animals will be shaved and prepped, and 10-mm wounds (bilateral) will be generated via a punch biopsy on the dorsal skin towards the scapular region. Following the wound excision, wounds will be treated with PLCs or PLCs covered with Tegaderm™ (3M) dressing. Wound dressing will be changed on the Days of wound measurement, i.e., Day 0, 1, 2, 3, 4, 5, 6, 7, 8, 9, 11, 12, and 14. On Day 0 immediately following wound induction and Day 1, 2, 3, 4, 5, 6, 7, 8, 9, 11, 12, and 14, wound sizes will be measured with a digital caliper (mm) on. The wound closure rates will also be calculated. Rats will be anesthetized to allow measurements to be taken. It is expected that PLCs will reduce the wound healing time in these rats.

### Example 3: PLC-Induced Hair Growth

Animals will be anesthetized with isoflurane and their backs will be shaved using an electrical razor to remove most of the hair in a single location while avoiding cutting or nicking the skin. The area of approximately 2 cm² will be shaved. Hair removal is conducted until the skin color is visible. Animals will be evaluated to determine if there is pigmentation present beneath the hair. If there is pigmentation, nicks, cuts or scars from fighting, these animals will be excluded from the study. Baseline pictures and scores will be taken for each rat after shaving and animals will be randomized based on skin color and initial BW. Hair growth scores will be taken twice per week, usually Mondays and Fridays, as well as weekly body weights and pictures throughout the course of the study.

Vehicle and Test Articles will be administered every 3 days SC to the shaved area while minoxidil (positive control) will be applied once per day topically according to study design (Table 1). For the topical application, 100 ul of solutions will be used for each application. For SC applications, test articles will be injected (100 ul) at the observation site. From these studies, it is expected that PLCs will facilitate hair growth.

TABLE 1

| | | | ROUTE/ | GROUP | DAYS OF | EVALUATIONS/ |
|---|---|---|---|---|---|---|
| GROUP | TREATMENT | DOSE | FREQUENCY | SIZE | DOSING | ENDPOINTS |
| 1 | Vehicle | 0 | SC every 3 days | 8 | 25 | Twice per week |
| 2 | Minoxidil | 5% | Topical QD | | | (e.g. Monday and |
| | | Solution | | | | Friday): |
| 3 | TA 1 | TBD | SC every 3 days | | | Clinical Hair |
| | Dose 1 | | | | | Growth Score |
| 4 | TA 1 | TBD | SC every 3 days | | | Weekly: |
| | Dose 2 | | | | | Body weight |
| 5 | TA 3 | TBD | SC every 3 days | | | Images (HG) |
| | Dose 3 | | | | | |

For hair growth evaluation rats were scored based upon hair growth using the scale below:

| SCORE | SCALE |
|---|---|
| 0 | Initial state-pink/grey skin with no hair growth |
| 0.5 | Skin color changes from pink/grey to bluish or contains |
| | dark pigment spots without visible hair growth |
| 1 | Skin color changes from pink/grey to bluish or contains dark |
| | pigment spots with visible hair growth (<20% coverage) |
| 1.5 | Grey/blue pigmentation with sparse hair growth (20-40% |
| | coverage) |
| 2 | Grey/blue pigmentation with sparse or diffuse short hair |
| | growth (40-60% coverage) |
| 2.5 | Grey/blue pigmentation with moderate hair growth |
| | (60-80% coverage) |
| 3 | Dense, normal coat hair (80-100% coverage) |

From the foregoing description, it will be apparent that variations and modifications may be made to the embodiments of the present disclosure to adopt it to various usages and conditions. Such embodiments are also within the scope of the following claims. The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1-48. (canceled)

49. A method of treating a condition in a subject, the method comprising: administering to the subject an effective amount of a composition comprising platelet-like cells (PLCs) or derivatives thereof or megakaryocyte-like cells (MLCS) or derivatives thereof, wherein a population of the PLCs or derivatives thereof or the MLCS or derivatives thereof comprise receptors of one or more of CD63>average2%, CD36<average80%, CD42b<average95%, or GPVI<average90% as compared to reference resting bone marrow derived platelet cells or megakaryocytes.

50. The method of claim 49, wherein the composition further comprises a wound healing agent, a tissue regeneration agent, an antiapoptotic agent, an anti-inflammatory agent, anti-hormonal agent, an immunomodulatory agent, extracellular vesicles (EV) or another therapeutic agent or a combination thereof.

51. The method of claim 49, wherein the composition is diluted to a physiological concentration in a carrier, wherein the carrier comprises a diluent or an excipient.

52. The method of claim 51, wherein the carrier is a plasma or a plasma substitute or a balanced crystalloid solution mimicking human plasma.

53. The method of claim 49, wherein the composition is administered to treat osteoarthritis, a wound healing or wound-healing related disorders, or a dry eye disease.

54. The method of claim 53, wherein the composition is administered to treat osteoarthritis.

55. The method of claim 53, wherein the composition is administered to treat dry eye disease.

56. The method of claim 49, wherein the PLCs, MLCs, or derivatives thereof are free of red blood cells or hemoglobin content or white blood cells.

57. The method of claim 49, wherein the composition further comprises extracellular vesicles (EV) ranging between 65 nm to 10 μm.

58. The method of claim 49, wherein the PLCs, MLCs, or derivatives thereof are generated by exposure of induced pluripotent stem cells (iPSCs) with one or more of shear stress, mechanical strain, or a pulsed electromagnetic field.

59. A composition as described by the method of claim 49, wherein the composition is one or more of: formulated for application to a site of injury for therapeutic use, lyophilized, implanted on an implantable device, cryopreserved, locally administered at a sight of or near an injury or a disease.

60. The composition of claim 59, wherein the formulation is performed in a buffer, diluent, or excipient or a combination thereof.

61. A method of treating a disease or disorder associated with injuries, the method comprising: administering an effective amount of a composition comprising PLCs, MLCs, or derivatives thereof to the subject, wherein the composition is administered in a therapeutic amount and wherein a reduction in injury recovery time is measured after administration of the composition and/or an improvement in pain, stiffness and function as measured after administration of the composition,
wherein a population of the PLCs, MLCs, or derivatives thereof comprise receptors of GPVI<average90% as compared to reference resting bone marrow derived platelet cells, and one or more of CD63>average2%, CD36<average80%, or CD42b<average95% as compared to reference resting bone marrow derived platelet cells.

62. The method according to claim 61, wherein the injury is osteoarthritis, a damaged tissue, a tendon injury, a ligament injury, a bone repair, a wound healing or wound-healing related disorder, dry eye disease, alopecia or an aging skin.

63. The method according to claim 61, wherein the composition further comprises a wound healing agent, a tissue regeneration agent, an antiapoptotic agent, an anti-inflammatory agent, anti-hormonal agent, an immunomodulatory agent or a combination thereof.

64. The method according to claim 63, wherein the tissue regeneration agent is a growth factors selected from one or more of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) and combinations thereof.

65. The method according to claim 61, wherein the composition is in a form of a gel, ointment, granules, tablet, suspension in a liquid carrier, capsule or powder.

66. The method according to claim 61, the composition comprises: a) between 1 and 100 wt % of PLCs, MLCs, or derivatives thereof or platelet-rich plasma derived therefrom, b) between 0 and 90 wt. % of a bulking agent, c) between 0 and 90 wt. % of at least one excipient or carrier, and optionally d) platelet-rich plasma derived from the subject.

67. A method of treating a dry eye disease, osteoarthritis, and a wound healing or wound-healing related disorder in a subject in need of such treatment comprising administering to the subject more than one dose of an effective amount of a composition comprising platelet-like cells (PLCs) or derivatives thereof, or megakaryocyte-like cells (MLCS) or derivatives thereof,
wherein a population of the PLCs, MLCs, or derivatives thereof comprise receptors of CD63>average2% as compared to reference resting bone marrow derived platelet cells, and one or more of CD36<average80%, CD42b<average95%, or GPVI<average90% as compared to reference resting bone marrow derived platelet cells or megakaryocytes.

68. The method according to claim 67, wherein the composition further comprises a wound healing agent, a tissue regeneration agent, an antiapoptotic agent, an anti-inflammatory agent, anti-hormonal agent or an immunomodulatory agent or a combination thereof.

69. The method according to claim 67, wherein the more than one dose is administered, daily, weekly, biweekly, triweekly or monthly.

70. The method according to claim 67, wherein an administration route is topical, transdermal, systemic, intravenous, intraarterial, intramuscular, subcutaneous, inhalation, rectal, buccal, vaginal, intraperitoneal, intraarticular, ophthalmic, otic, or oral.

71. A composition for treating a condition in a subject, the composition made according to the method of claim 49.
